(19) United States
(12) Patent Application Publication
(10) Pub. No.: US 2008/0188659 A1
(43) Pub. Date: Aug. 7, 2008

(54) Method for Preparing Halogenated Amines

(75) Inventors: Eddine Saiah, Brookline, MA (US); Huan-Qiu Li, Brighton, MA (US); Adrian Huang, Lexington, MA (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 12/027,412

(22) Filed: Feb. 7, 2008

Related U.S. Application Data
(60) Provisional application No. 60/900,261, filed on Feb. 7, 2007.

(51) Int. Cl. C07D 241/04; C07C 311/16; C07C 311/21; C07D 211/60

(52) U.S. Cl. 544/383; 564/90; 564/91; 546/226

(57) ABSTRACT

This invention relates to methods for preparing halogenated amines.

### CROSS REFERENCE TO RELATED APPLICATIONS

This application claims the benefit of U.S. Provisional Application No. 60/900,261, filed on Feb. 7, 2007, which is incorporated herein by reference in its entirety.

### TECHNICAL FIELD

This invention relates to methods for preparing halogenated amines.

### BACKGROUND

The replacement of one or more hydrogen atoms in an organic molecule with one or more fluorine atoms can sometimes alter its chemical and biological nature, including its stability, lipophilicity, and bioavailability. C—F bond is known to mimic a C—H bond because of its similar bond length, and fluorinated groups are known to be isosteres of many common substituents. Trifluoromethyl group (CF₃) containing compounds are known to have applications in the materials field, as well as in the pharmaceutical and agrochemical industries.

### SUMMARY

This invention relates generally to methods for preparing compounds containing one or more halogenated amines (e.g., bis(trifluoromethylated) amines) from nitrile-containing starting materials and intermediates. The inventors have discovered that reacting a nitrile (i.e., C≡N, also referred to as a cyano group) containing organic compound with a fluoroalkyl (R^{F}) transfer agent (also referred to herein as a fluoroalkylating agent) results in the conversion of the nitrile to an amine. This process is summarized in the nonlimiting scheme below:

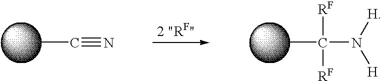

The shaded circle represents the organic compound, and each R^{F} is a fluoroalkyl group (e.g., CF₃).

In one aspect, this invention features a method for preparing an organic compound having one or more (e.g., 1, 2, 3, 4, 5, or 6, e.g., 1 or 2) substituents of formula (A):

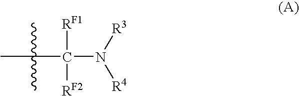

in which:

(i) each of R^{F1} and R^{F2} can be, independently, optionally substituted C₁-C₆ fluoroalkyl (e.g., C₁-C₄ perfluoroalkyl, e.g., CF₃), e.g., optionally substituted with from 1-2 substituents as described herein;

(ii) each of R³ and R⁴ can be, independently, hydrogen, R^{a}, —C(O)H, —C(O)R^{a}, —C(O)OR^{a}, or —SO₂R^{a}, wherein R^{a} at each occurrence can be, independently, any organic group, e.g., alkyl, cycloalkyl, aralkyl, heterocyclyl, aryl, or heteroaryl, each of which can be optionally substituted as described herein; e.g., C₁-C₁₂ (e.g., C₁-C₁₀, C₁-C₆, or C₁-C₄) alkyl, C₃-C₁₀ (e.g., C₃-C₈, C₃-C₆) cycloalkyl, C₇-C₂₀ (e.g., C₇-C₁₆, C₇-C₁₂, C₇-C₁₀) aralkyl, heterocyclyl including 3-10 (e.g., 3-8, 3-6) atoms, C₆-C₁₈ (e.g., C₆-C₁₄, C₆-C₁₀, or phenyl) aryl, or heteroaryl including 5-16 (e.g., 5-12, 5-10, or 5-6) atoms, each of which can be optionally substituted with from 1-10 (e.g., 1-5, 1-4, 1-3, 1-2, or 1) substituents as described herein; and

(iii) the organic compound can include as part of its structure any one or more of the following substructures:

(i) C₆-C₁₈ aryl or heteroaryl including 5-16 atoms, each of which is optionally substituted; e.g., C₆-C₁₈ (e.g., C₆-C₁₄, C₆-C₁₀, or phenyl) aryl or heteroaryl including 5-16 (e.g., 5-12, 5-10, or 5-6) atoms, each of which can be optionally substituted with from 1-10 (e.g., 1-5, 1-4, 1-3, 1-2, or 1) substituents as described herein;

(ii) C₇-C₂₀ aralkyl or heteroaralkyl including 6-20 atoms, each of which is optionally substituted; e.g., C₇-C₂₀ (e.g., C₇-C₁₆, C₇-C₁₂, C₇-C₁₀) aralkyl or heteroaralkyl including 6-20 (e.g., 6-14 or 6-10) atoms, each of which can be optionally substituted with from 1-10 (e.g., 1-5, 1-4, 1-3, 1-2, or 1) substituents as described herein; or

(iii) C₃-C₁₀ cycloalkyl, C₃-C₁₀ cycloalkenyl, heterocyclyl including 3-10 atoms, or heterocycloalkenyl including 3-10 atoms, each of which is optionally substituted; e.g., C₃-C₁₀ (e.g., C₃-C₈, C₃-C₆) cycloalkyl, C₃-C₁₀ (e.g., C₃-C₈, C₃-C₆) cycloalkenyl, heterocyclyl including 3-10 (e.g., 3-8, 3-6) atoms, or heterocycloalkenyl including 3-10 (e.g., 3-8, 3-6) atoms, each of which can be optionally substituted with from 1-5 (e.g., 1-4, 1-3, 1-2, or 1) substituents as described herein; or

(iv) C₁-C₁₂ alkyl, C₂-C₁₂ alkenyl or C₂-C₁₂ alkynyl, each of which is optionally substituted; e.g., C₁-C₁₂ (e.g., C₁-C₁₀, C₁-C₆, or C₁-C₄) alkyl, C₂-C₁₂ (e.g., C₂-C₁₀, C₂-C₆, or C₂-C₄) alkenyl or C₂-C₁₂ (e.g., C₂-C₁₀, C₂-C₆, or C₂-C₄) alkynyl, each of which can be optionally substituted with from 1-5 (e.g., 1-4, 1-3, 1-2, or 1) substituents as described herein;

each of which (when two or more substructures are present) can be connected to one another via one (or more) direct bonds or heteroatom-containing linker(s) (e.g., SO₂), or any combination thereof.

The method includes reacting one or more nitrile-containing organic compounds (the terms “organic compound” and “compound” will be used interchangeably throughout this specification) with one or more fluoroalkylating agents.

In some embodiments, the method can be used to prepare compounds having one substituent of formula A. In these embodiments, the method can include reacting the corresponding nitrile substituted compound with a fluoroalkylating agent. The starting material, intermediates, and/or product can include one or more of the substructures described herein.

In some embodiments, the method can be used to prepare compound having two or more (e.g., 2, 3, 4, 5, or 6, e.g., 2) substituents of formula A.

In certain embodiments, when the organic compound includes two or more substructures (e.g., an aryl ring; a heterocyclic ring; and either a heteroaryl ring or a second aryl ring), each of the substituents of formula A can be located on the same substructure, or each of the substituents of formula A can be distributed among two or more of the substructures.

In certain embodiments, each of the substituents of formula A can be introduced in the same reaction step. For example, a compound having two substituents of formula A can be prepared by reacting a starting material having two nitrile groups with an appropriate amount of the fluoroalkylating agent.

In certain embodiments, each of the substituents of formula A can be introduced sequentially. See, e.g., the nonlimiting scheme below:

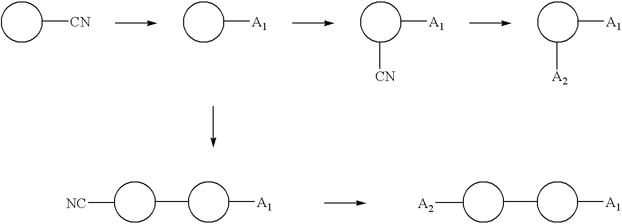

The open circles represent an organic compound or a substructure thereof, A₁ and A₂ each represent a substituent of formula A (each of which can be the same or different); and CN represents a nitrile group.

In another aspect, this invention features a method for preparing a compound of formula (I-A) or a salt thereof from a compound of formula (II-A).

The structure of formula (II-A) is shown below:

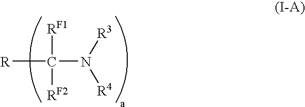

in which:

a is 1, 2, 3, 4, 5, or 6 (e.g., 1 or 2, e.g., 1);

R is:

(i) C₆-C₁₈ aryl or heteroaryl including 5-16 atoms, each of which is optionally substituted; e.g., C₆-C₁₈ (e.g., C₆-C₁₄, C₆-C₁₀, or phenyl) aryl or heteroaryl including 5-16 (e.g., 5-12, 5-10, or 5-6) atoms, each of which can be optionally substituted with from 1-10 (e.g., 1-5, 1-4, 1-3, 1-2, or 1) substituents as described herein; or

(ii) C₇-C₂₀ aralkyl or heteroaralkyl including 6-20 atoms, each of which is optionally substituted; e.g., C₇-C₂₀ (e.g., C₇-C₁₆, C₇-C₁₂, C₇-C₁₀) aralkyl or heteroaralkyl including 6-20 (e.g., 6-14 or 6-10) atoms, each of which can be optionally substituted with from 1-10 (e.g., 1-5, 1-4, 1-3, 1-2, or 1) substituents as described herein; or

(iii) C₃-C₁₀ cycloalkyl, C₃-C₁₀ cycloalkenyl, heterocyclyl including 3-10 atoms, or heterocycloalkenyl including 3-10 atoms, each of which is optionally substituted; e.g., C₃-C₁₀ (e.g., C₃-C₈, C₃-C₆) cycloalkyl, C₃-C₁₀ (e.g., C₃-C₈, C₃-C₆) cycloalkenyl, heterocyclyl including 3-10 (e.g., 3-8, 3-6) atoms, or heterocycloalkenyl including 3-10 (e.g., 3-8, 3-6) atoms, each of which can be optionally substituted with from 1-5 (e.g., 1-4, 1-3, 1-2, or 1) substituents as described herein; or

(iv) C₁-C₁₂ alkyl, C₂-C₁₂ alkenyl or C₂-C₁₂ alkynyl, each of which is optionally substituted; e.g., C₁-C₁₂ (e.g., C₁-C₁₀, C₁-C₆, or C₁-C₄) alkyl, which can be optionally substituted with from 1-5 (e.g., 1-4, 1-3, 1-2, or 1) substituents as described herein; or C₂-C₁₂ (e.g., C₂-C₁₀, C₂-C₆, or C₂-C₄) alkenyl or C₂-C₁₂ (e.g., C₂-C₁₀, C₂-C₆, or C₂-C₄) alkynyl, each of which can be optionally substituted with from 1-5 (e.g., 1-4, 1-3, 1-2, or 1) substituents as described herein;

each of R^{F1} and R^{F2} is, independently, optionally substituted C₁-C₆ fluoroalkyl, e.g., optionally substituted with from 1-2 substituents as described herein; and

each of R³ and R⁴ is, independently, hydrogen, R^{a}, —C(O)H, —C(O)R^{a}, —C(O)OR^{a}, or —SO₂R^{a}, wherein R^{a} at each occurrence is, independently, as defined above for R^{a}.

The structure of formula (II-A) is shown below:

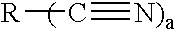

(II-A); (R and a can be as defined above for formula (I-A)).

The method includes reacting the compound of formula (II-A) with a fluoroalkylating agent. In these embodiments, when a>1, then the nitrile groups in formula (II-A) and substituents of formula A in formula (I-A) can be located anywhere along R. For example, if R is an aryl group that is substituted with, e.g., a heterocyclic ring that itself is further substituted, e.g., with another cyclic structure, then the nitrile groups in formula (II-A) and substituents of formula A in formula (I-A) can be present on the base substituent (here, an aryl group) and/or any substituent thereof (e.g., the heterocyclic ring and/or the other cyclic structure).

In a further aspect, this invention features a method for preparing a compound of formula (I) or a salt thereof from a compound of formula (II).

The structure of formula (I) is shown below:

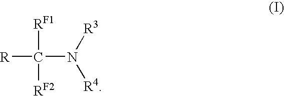

The structure of formula (II) is shown below:

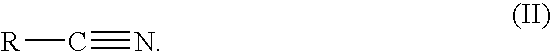

R, R^{F1}, R^{F2}, R³, and R⁴ can be as defined above for formulas (I-A) and (II-A).

The method includes reacting the compound of formula (II) with a fluoroalkylating agent.

In one aspect, this invention features a method for preparing a compound of formula (I) or (I-A) or a salt thereof from a compound of formula (II) or (II-A), respectively, in which R in formulas (I), (I-A), (II), and (II-A) can be C₆-C₁₀ aryl or heteroaryl including 5-10 atoms, each of which is:

(a) substituted with 1-SO₂NR^{N1}R^{N2} or —C(O)NR^{N1}R^{N2}; and

(b) optionally further substituted with from 1-5 substituents as described herein.

Each of R^{N1} and R^{N2} can be, independently of one another:

(i) hydrogen; or

(ii) C₁-C₁₂ (e.g., C₁-C₁₀, C₁-C₆, or C₁-C₄) alkyl or C₁-C₁₂ (e.g., C₁-C₁₀, C₁-C₆, or C₁-C₄) haloalkyl; each of which is optionally substituted with from 1-5 (e.g., 1-4, 1-3, 1-2, or 1) substituents as described herein; or

(iii) C₇-C₁₂ (e.g., C₇-C₁₀, benzyl) aralkyl; C₃-C₁₀ (e.g., C₃-C₈, C₃-C₆) cycloalkyl; heteroaralkyl including 6-12 (e.g., 6-10) atoms; C₃-C₁₀ (e.g., C₃-C₈, C₃-C₆) cycloalkenyl; heterocyclyl including 3-10 (e.g., 3-8, 3-6) atoms; or heterocycloalkenyl including 3-10 (e.g., 3-8, 3-6) atoms; each of which is optionally substituted with from 1-5 (e.g., 1-4, 1-3, 1-2, or 1) substituents as described herein; or

(iv) C₂-C₁₀ alkenyl or C₂-C₁₀ alkynyl, each of which is optionally substituted with from 1-5 (e.g., 1-4, 1-3, 1-2, or 1) substituents as described herein; or

(v) C₆-C₁₀ aryl or heteroaryl including 5-10 atoms, each of which is optionally substituted with from 1-5 (e.g., 1-4, 1-3, 1-2, or 1) substituents as described herein; or

(vi) —C(O)H, —C(O)R^{a}, —C(O)OR^{a}, or —SO₂R^{a}, in which R^{a} can be as defined anywhere herein; or

(vii) R^{N1} and R^{N2}, together with the nitrogen atom to which each is attached, form a heterocyclyl including 3-10 (e.g., 3-8, 3-6, 5-6) atoms, which is optionally substituted with from 1-5 (e.g., 1-4, 1-3, 1-2, or 1) substituents as described herein. The heterocyclyl can further include one or more (e.g., 1 or 2) heteroatoms, e.g., nitrogen or oxygen, in addition to the nitrogen atom to which each of R^{N1} and R^{N2} is attached. When the additional heteroatom is a nitrogen atom, this additional nitrogen atom can be attached to a hydrogen atom or a substituent other than hydrogen as described herein.

In certain embodiments, each of R^{N1} and R^{N2} can be, independently, a substituent other than hydrogen. In these embodiments, R^{N1} and R^{N2} can be the same substituent or each can be a different substituent.

For example, each of R^{N1} and R^{N2} can be independently of one another unsubstituted C₁-C₁₂ (e.g., C₁-C₁₀, C₁-C₆, or C₁-C₄) alkyl.

As another example, one of R^{N1} and R^{N2} can be selected from (ii)-(v) above, e.g.:
C₇-C₁₂ (e.g., C₇-C₁₀, benzyl) aralkyl, which is substituted with from 1-5 (e.g., 1-4, 1-3, 1-2, or 1) substituents as described herein; or
C₃-C₁₀ (e.g., C₃-C₈, C₃-C₆) cycloalkyl, which is substituted with from 1-5 (e.g., 1-4, 1-3, 1-2, or 1) substituents as described herein; or
C₆-C₁₀ aryl or heteroaryl including 5-10 atoms, each of which is optionally substituted with from 1-5 (e.g., 1-4, 1-3, 1-2, or 1) substituents as described herein;
   and the other can be —C(O)H, —C(O)R^{a}, or —C(O)OR^{a} (e.g., —C(O)OR^{a}, in which R^{a} is an unsubstituted C₁-C₄ alkyl).

As a further example, R^{N1} and R^{N2}, together with the nitrogen atom to which each is attached, form a heterocyclyl including 5 or 6 atoms (e.g., piperidyl (piperidino), piperazinyl, morpholinyl (morpholino), pyrrolinyl, and pyrrolidinyl), which can be optionally substituted with from 1-5 (e.g., 1-4, 1-3, 1-2, or 1) substituents as described herein. In certain embodiments, the heterocyclyl is unsubstituted. In other embodiments, the heterocyclyl is substituted with other than optionally substituted aryl, optionally substituted heteroaryl, optionally substituted cycloalkyl, optionally substituted cycloalkenyl, optionally substituted heterocycloalkenyl, or is not directly substituted with an oxygen, sulfur or nitrogen atom. In still other embodiments, the heterocyclyl can be substituted with from 1-5 C₁-C₆ alkyl groups.

In certain embodiments, R can be:

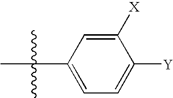

in which one of X and Y is —SO₂NR^{N1}R^{N2} or —C(O)NR^{N1}R^{N2}, and the other is hydrogen. In these embodiments, each of R^{N1} and R^{N2} can be, independently of one another, as defined anywhere herein.

In one aspect, this invention features a method for preparing a compound of formula (VII) or a salt thereof from a compound of formula (VIII).

The structure of formula (VII) is shown below:

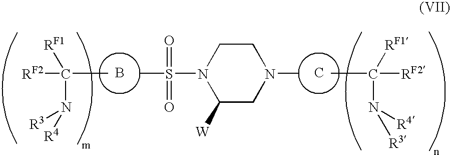

in which:

each of m and n is, independently, 0, 1, 2, or 3 (e.g., 0, 1, or 2, e.g., 0 or 1), provided that one of m and n is 1;

each of R^{F1}, R^{F2}, R^{F1′}, and R^{F2′} is, independently, optionally substituted C₁-C₆ fluoroalkyl, e.g., optionally substituted with from 1-2 substituents as described herein;

each of R³, R⁴, R^{3′}, and R^{4′} is, independently, hydrogen, C₁-C₆ alkyl, —C(O)H, or —C(O)OR^{a}, wherein R^{a} is C₇-C₂₀ aralkyl (e.g., benzyl or fluorenyl) or C₁-C₆ alkyl (e.g., tert-butyl), each of which is optionally substituted, e.g., with from 1-3 substituents as described herein;

ring B is C₆-C₁₀ aryl or heteroaryl including 5-10 atoms, each of which is optionally further substituted with from 1-5 substituents independently selected from halo; NR^{f}R^{g}; hydroxyl; C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted, e.g., with from 1-5 substituents as described herein; optionally substituted C₁-C₁₂ (e.g., C₁-C₁₀, C₁-C₆, C₁-C₄, or C₁-C₃) alkoxy, e.g., with from 1-5 substituents as described herein; C₁-C₁₂ (e.g., C₁-C₁₀, C₁-C₆, C₁-C₄, or C₁-C₃) haloalkoxy; nitro; C₆-C₁₀ aryl or heteroaryl including 5-12 atoms, each of which is optionally substituted, e.g., with from 1-5 substituents as described herein; C₆-C₁₀ aryloxy or heteroaryloxy including 5-12 (e.g., 5-10 or 5-6) atoms, each of which is optionally substituted, e.g., with from 1-5 substituents as described herein; heterocyclyl including 3-10 (e.g., 3-6 or 5-6) atoms, C₃-C₁₀ (e.g., C₃-C₈, C₃-C₆) cycloalkyl, C₇-C₁₂ aralkoxy or heteroaralkoxy including 6-12 atoms, each of which is optionally substituted, e.g., with from 1-5 substituents as described herein; —C(O)OR^{h}; —C(O)NR^{f}R^{g}; or —NRⁱC(O)R^{j};

each of R^{f}, R^{g}, and R^{h}, at each occurrence is, independently:

(i) hydrogen; or

(ii) C₁-C₁₂ alkyl or C₁-C₁₂ haloalkyl; each of which is optionally substituted; e.g., C₁-C₁₂ (e.g., C₁-C₁₀, C₁-C₆, or C₁-C₄) alkyl or C₁-C₁₂ (e.g., C₁-C₁₀, C₁-C₆, or C₁-C₄) haloalkyl; each of which is optionally substituted with from 1-5 (e.g., 1-4, 1-3, 1-2, or 1) substituents as described herein; or

(iii) C₇-C₂₀ aralkyl; C₃-C₁₆ cycloalkyl; heteroaralkyl including 6-20 atoms; C₃-C₁₆ cycloalkenyl; heterocyclyl including 3-16 atoms; or heterocycloalkenyl including 3-16 atoms; each of which is optionally substituted; e.g., C₇-C₂₀ (e.g., C₇-C₁₆, C₇-C₁₂, C₇-C₁₀) aralkyl; C₃-C₁₆ (e.g., C₃-C₁₀, C₃-C₈, C₃-C₆) cycloalkyl; heteroaralkyl including 6-20 (e.g., 6-14, 6-10) atoms; C₃-C₁₆ (e.g., C₃-C₁₀, C₃-C₈, C₃-C₆) cycloalkenyl; heterocyclyl including 3-16 (e.g., 3-10, 3-8, 3-6) atoms; or heterocycloalkenyl including 3-16 (e.g., 3-10, 3-8, 3-6) atoms; each of which is optionally substituted with from 1-10 (e.g., 1-5, 1-4, 1-3, 1-2, or 1) substituents as described herein; or

(iv) C₂-C₂₀ (e.g., C₂-C₁₂, C₂-C₁₀, C₂-C₆, or C₂-C₄) alkenyl or C₂-C₂₀ (e.g., C₂-C₁₂, C₂-C₁₀, C₂-C₆, or C₂-C₄) alkynyl; or

(v) C₆-C₁₆ aryl or heteroaryl including 5-16 atoms, each of which is optionally substituted; e.g., C₆-C₁₆ (e.g., C₆-C₁₄, C₆-C₁₀, or phenyl) aryl or heteroaryl including 5-16 (e.g., 5-12, 5-10, or 5-6) atoms, each of which is optionally substituted with from 1-10 (e.g., 1-5, 1-4, 1-3, 1-2, or 1) substituents as described herein; or optionally

(vi) —C(O)H, —C(O)R^{a}, —C(O)OR^{a}, or —SO₂R^{a}, in which R^{a} can be as defined anywhere herein;

Rⁱ is hydrogen or unsubstituted C₁-C₃ alkyl;

R^{j} is R^{h}; OR^{h}; or NR^{f}R^{g};

W is C₁-C₄ alkyl; and

ring C is C₆-C₁₀ aryl or heteroaryl including 5-10 atoms, each of which is optionally further substituted with from 1-5 substituents independently selected from halo; C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted, e.g., with from 1-5 (e.g., 1-4, 1-3, 1-2, or 1) substituents as described herein; C₁-C₁₂ (e.g., C₁-C₁₀, C₁-C₆, C₁-C₄, or C₁-C₃) alkoxy; C₁-C₁₂ (e.g., C₁-C₁₀, C₁-C₆, C₁-C₄, or C₁-C₃) haloalkoxy; nitro; or C₆-C₁₀ aryl or heteroaryl including 5-12 (e.g., 5-10) atoms, each of which is optionally substituted, e.g., with from 1-5 (e.g., 1-4, 1-3, 1-2, or 1) substituents as described herein.

The structure of formula (VIII) is shown below:

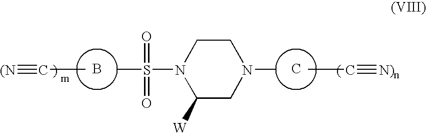

in which:

ring B is C₆-C₁₀ aryl or heteroaryl including 5-10 atoms, each of which is optionally further substituted with from 1-5 substituents independently selected from halo; NR^{f}R^{g}; hydroxyl; C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted, e.g., with from 1-5 (e.g., 1-4, 1-3, 1-2, or 1) substituents described herein; optionally substituted C₁-C₁₂ (e.g., C₁-C₁₀, C₁-C₆, C₁-C₄, or C₁-C₃) alkoxy, e.g., with from 1-5 (e.g., 1-4, 1-3, 1-2, or 1) substituents described herein; C₁-C₁₂ (e.g., C₁-C₁₀, C₁-C₆, C₁-C₄, or C₁-C₃) haloalkoxy; nitro; C₆-C₁₀ aryl or heteroaryl including 5-12 atoms, each of which is optionally substituted, e.g., with from 1-5 (e.g., 1-4, 1-3, 1-2, or 1) substituents described herein; C₆-C₁₀ aryloxy or heteroaryloxy including 5-12 atoms, each of which is optionally substituted with from 1-5 (e.g., 1-4, 1-3, 1-2, or 1) substituents described herein; heterocyclyl including 3-10 (e.g., 3-6 or 5-6) atoms, C₃-C₁₀ (e.g., C₃-C₈, C₃-C₆) cycloalkyl, C₇-C₁₂ aralkoxy or heteroaralkoxy including 6-12 atoms, each of which is optionally substituted, e.g., with from 1-5 (e.g., 1-4, 1-3, 1-2, or 1) substituents described herein; —C(O)R^{e}, wherein R^{e} is C₁-C₆ alkyl; —C(O)NR^{f}R^{g}; or —NRⁱC(O)R^{j}; and

R^{f}, R^{g}, R^{h}, Rⁱ, R^{j}, m, n, ring C, and W can be as defined above in conjunction with formula (VII).

The method includes reacting the compound of formula (VIII) with a fluoroalkylating agent.

In some embodiments, the methods described herein can be used to prepare compounds that modulate (e.g., inhibit) 11βHSD1.

In one aspect, this invention features the compounds themselves of formulas (I), (I-A), and (VII), including any subgenus or specific compound(s) thereof, and/or pharmaceutically acceptable salts thereof. In an embodiment, the compound can be selected from the group consisting of:
2-(3-{[(2R)-4-{6-[1-amino-2,2,2-trifluoro-1-(trifluoromethyl)ethyl]pyridin-3-yl}-2-methylpiperazin-1-yl]sulfonyl}phenyl)-1,1,1-trifluoropropan-2-ol;
2-(3-{[(2R)-4-{4-[1-amino-2,2,2-trifluoro-1-(trifluoromethyl)ethyl]-2-(trifluoromethyl)phenyl}-2-methylpiperazin-1-yl]sulfonyl}phenyl)-1,1,1-trifluoropropan-2-ol;
2-[4-({(2R)-4-[4-[1-amino-2,2,2-trifluoro-1-(trifluoromethyl)ethyl]-2-(trifluoromethyl)phenyl]-2-methylpiperazin-1-yl}sulfonyl)phenyl]-1,1,1-trifluoropropan-2-ol;
1,1,1,3,3,3-hexafluoro-2-[4-({(2R)-4-[4-fluoro-2-(trifluoromethyl)phenyl]-2-methylpiperazin-1-yl}sulfonyl)phenyl]propan-2-amine; and
1,1,1,3,3,3-hexafluoro-2-[3-({(2R)-4-[4-fluoro-2-(trifluoromethyl)phenyl]-2-methylpiperazin-1-yl}sulfonyl)phenyl]propan-2-amine; or a_pharmaceutically acceptable salt thereof.

In another embodiment, the compound can be selected from the group consisting of the title compounds of Examples 1-13.

In one aspect, this invention features a pharmaceutical composition, which includes a compound of formulas (I), (I-A), or (VII), including any subgenus or specific compound(s) thereof, or a salt (e.g., a pharmaceutically acceptable salt) thereof, or a prodrug thereof (e.g., an effective amount thereof); and a pharmaceutically acceptable adjuvant, carrier or diluent.

In another aspect, this invention features a method of preparing a pharmaceutical composition that includes admixing a compound of formula (I), (I-A), or (VII), including any subgenus or specific compound(s) thereof, or a salt (e.g., a pharmaceutically acceptable salt) thereof, or a prodrug thereof (e.g., an effective amount thereof) with a pharmaceutically acceptable adjuvant, carrier or diluent.

In one aspect, this invention relates to a method for treating a disease or condition mediated by excess or uncontrolled amounts of cortisol and/or other corticosteroids, which includes administering to a subject in need thereof an effective amount of a compound of formula (I), (I-A), or (VII), including any subgenus or specific compound(s) thereof, or a salt (e.g., a pharmaceutically acceptable salt) thereof, or a prodrug thereof.

In another aspect of the invention, this invention relates to methods for treating, controlling, ameliorating, preventing, delaying the onset of, or reducing the risk of developing one or more of diabetes (e.g., type 1 or type 2 diabetes), Syndrome X, hyperglycemia, low glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, pancreatitis, abdominal obesity, neurodegenerative disease, retinopathy, nephropathy, neuropathy, hypertension, coronary heart disease, stroke, peripheral vascular disease, Cushing's syndrome, glaucoma, osteoperosis, hyperinsulinemia, tuberculosis, psoriasis, cognitive disorders and dementia (e.g., impairment associated with aging and of neuronal dysfunction, e.g., Alzheimer's disease), depression, viral diseases, inflammatory disorders, immune disorders); or promoting wound healing, which includes administering to a subject in need thereof an effective amount of a compound of formula (I), (I-A), or (VII), including any subgenus or specific compound(s) thereof, or a salt (e.g., a pharmaceutically acceptable salt) thereof or a prodrug thereof.

Embodiments can include one or more of the following features.

The fluoroalkylating agent can have any one of the formulae delineated herein.

R^{F1} and R^{F2} can be the same or different. Each of R^{F1} and R^{F2} can be, independently, optionally substituted C₁-C₄ perfluoroalkyl (e.g., CF₃).

Each of R³ and R⁴ can be hydrogen.

R can be optionally substituted C₆-C₁₀ aryl (e.g., optionally substituted phenyl), e.g., optionally substituted with from 1-5 (e.g., 1-4, 1-3, 1-2, or 1) substituents as described herein. R can be optionally substituted C₇-C₁₂ aralkyl (e.g., benzyl), e.g., optionally substituted with from 1-5 (e.g., 1-4, 1-3, 1-2, or 1) substituents as described herein.

m in formulas (VII) and (VIII) can be 1, and n in formulas (VII) and (VIII) can be 0. In embodiments, each of R^{F1} and R^{F2} in formula (VII) can be CF₃. Each of R³ and R⁴ in formula (VII) can be hydrogen. Ring C in formula (VII) has formula (IX):

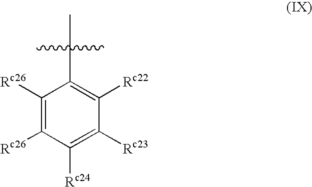

in which two of R^{c22}, R^{c23}, R^{c24}, R^{c25}, and R^{c26} can each be, independently, halo; C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted, e.g., with from 1-5 (e.g., 1-4, 1-3, 1-2, or 1) substituents as described herein; C₁-C₁₂ (e.g., C₁-C₁₀, C₁-C₆, C₁-C₄, or C₁-C₃) alkoxy; C₁-C₁₂ (e.g., C₁-C₁₀, C₁-C₆, C₁-C₄, or C₁-C₃) haloalkoxy; nitro; or C₆-C₁₀ aryl or heteroaryl including 5-12 atoms, each of which is optionally substituted, e.g., with from 1-5 (e.g., 1-4, 1-3, 1-2, or 1) substituents as described herein; and the others are hydrogen. R^{c22} can be CF₃ or fluoro; and R^{c24} can be fluoro, chloro, CF₃, or optionally substituted heteroaryl.

m in formulas (VII) and (VIII) can be 0, and n in formulas (VII) and (VIII) can be 1. In embodiments, each of R^{F1′} and R^{F2′} in formula (VII) can be CF₃. Each of R^{3′} and R^{4′} in formula (VII) can be hydrogen. Ring B in formula (VII) can have formula (X):

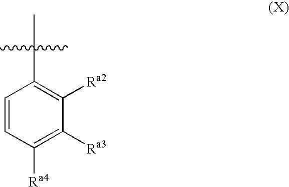

wherein one of R^{a2}, R^{a3}, and R^{a4} is halo; NR^{f}R^{g}; hydroxyl; C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted, e.g., with from 1-5 substituents as described herein; optionally substituted C₁-C₁₂ (e.g., C₁-C₁₀, C₁-C₆, C₁-C₄, or C₁-C₃) alkoxy, e.g., optionally substituted with from 1-5 (e.g., 1-4, 1-3, 1-2, or 1) substituents as described herein; C₁-C₁₂ (e.g., C₁-C₁₀, C₁-C₆, C₁-C₄, or C₁-C₃) haloalkoxy; nitro; C₆-C₁₀ aryl or heteroaryl including 5-12 atoms, each of which is optionally substituted, e.g., with from 1-5 (e.g., 1-4, 1-3, 1-2, or 1) substituents as described herein; C₆-C₁₀ aryloxy or heteroaryloxy including 5-12 atoms, each of which is optionally substituted, e.g., with from 1-5 (e.g., 1-4, 1-3, 1-2, or 1) substituents as described herein; heterocyclyl including 3-10 atoms, C₃-C₁₀ (e.g., C₃-C₆) cycloalkyl, C₇-C₁₂ aralkoxy or heteroaralkoxy including 6-12 atoms, each of which is optionally substituted, e.g., with from 1-5 (e.g., 1-4, 1-3, 1-2, or 1) substituents as described herein; —C(O)OR^{h}; —C(O)NR^{f}R^{g}; or —NRⁱC(O)R^{j}; and the others are hydrogen. R^{a3} or R^{a4} can be 1,1,1-trifluoro-2-hydroxy-2-propyl (e.g., R, S, or R and S configuration at the carbon attached to the hydroxyl group). In some embodiments, ring B in formula (VIII) can be substituted with —C(O)R^{e}, wherein R^{e} is C₁-C₄ alkyl. In certain embodiments, the —C(O)R^{e} can be present when the nitrile group is allowed to react with the fluoroalkylating agent.

The starting materials, intermediates, and products can be S or N-oxides and/or salts (e.g., pharmaceutically acceptable salts) thereof.

The methods can further include forming a salt (e.g., a pharmaceutically acceptable salt) and/or admixing the compound with a pharmaceutically acceptable adjuvant, carrier or diluent. The methods can further include the separation of stereoisomer products or starting materials.

The listing of permissible optional substituents for a starting material (e.g., nitrile-containing organic compound as described herein) can be different from that for a product (e.g., organic compound containing substituents having formula (A) as described herein). For example, the starting material can be only further substituted (i.e., in addition to the nitrile) with moieties known to be stable or inert to a particular fluoroalkylating agent or classes thereof (e.g., the fluoroalkylating agents described herein). Thus, in some embodiments, the methods can further include the introduction of substituents to a particular (specific or generic) nitrile-containing starting material or to a particular (specific or generic) compound containing substituents having formula (A). The methods can further include the modification (e.g., deprotections) of substituents that may be present on a particular (specific or generic) nitrile-containing starting material or to a particular (specific or generic) compound containing substituents having formula (A). Such processes include, but are not limited to, those described in US 2007-0219198, filed on Feb. 7, 2007, which is incorporated herein by reference in its entirety.

The term “fluoroalkyl” refers to an alkyl group, in which one or more hydrogen atoms is replaced by fluorine atom (F). In some embodiments, more than one hydrogen atom (e.g., 2, 3, 4, 5, 6, 7, 8, 9, 10, 11, 12, 13, etc. hydrogen atoms) on a alkyl group can be replaced by more than one fluorine atom (2, 3, 4, 5, 6, 7, 8, 9, 10, 11, 12, or 13 fluorine atoms). The term “fluoroalkyl” also includes alkyl moieties in which all of hydrogen atoms have been replaced by fluorine atoms (e.g., sometimes referred to as perfluoroalkyl moieties, such as trifluoromethyl).

The term “fluoroalkylating agent” refers to:

(1) a fluoroalkyl-containing, nonionic compound, which upon interaction with (i) a catalytic, stoichiometric, or excess amount of a neutral or charged chemical entity; (ii) light; (iii) heat; or (iv) any combination thereof, fully or partially dissociates to produce the corresponding fluoroalkyl carbanion or radical, or a reactive equivalent thereof, or

(2) a fluoroalkyl-containing salt or ionic complex.

The term “halo” or “halogen” refers to any radical of fluorine, chlorine, bromine or iodine. The term “carboxy” refers to the —COOH radical.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be optionally substituted, e.g., with one or more substituents. Examples of alkyl groups include without limitation methyl, ethyl, and tert-butyl.

The term “cycloalkyl” refers to saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl groups can contain fused rings. Fused rings are rings that share a common carbon atom. Cycloalkyl moieties can include, e.g., cyclopropyl, cyclohexyl, methylcyclohexyl (provided that the methylcyclohexyl group is attached to another moiety via a cyclohexyl ring carbon and not the methyl group), adamantyl, and norbornyl (bicyclo[2.2.1]heptyl).

The term “haloalkyl” refers to an alkyl group, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on a alkyl group can be replaced by more than one halogen (e.g., 2, 3, 4, 5, 6, 7, 8, 9, 10, 11, 12, 13, 14, 15, 16, 17, 18, 19, 20, 21, 22, 23, 24, 25, 26, etc. halogen atoms). In these embodiments, the hydrogen atoms can each be replaced by the same halogen (e.g., fluoro) or the hydrogen atoms can be replaced by a combination of different halogens (e.g., fluoro and chloro). The term “haloalkyl” also includes alkyl moieties in which all hydrogens have been replaced by halo (e.g., sometimes referred to as perhaloalkyl moieties, such as trifluoromethyl). The term “fluoroalkyl” defined above is a subset of haloalkyl.

The term “aralkyl” refers to an alkyl moiety in which an alkyl hydrogen atom is replaced by an aryl group. One of the carbons of the alkyl moiety serves as the point of attachment of the aralkyl group to another moiety. Aralkyl includes groups in which more than one hydrogen atom on an alkyl moiety has been replaced by an aryl group. Any ring or chain atom can be optionally substituted e.g., by one or more substituents. Examples of “aralkyl” include without limitation benzyl, 2-phenylethyl, 3-phenylpropyl, benzhydryl (diphenylmethyl), and trityl (triphenylmethyl) groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be optionally substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be optionally substituted, e.g., by one or more substituents. Alkenyl groups can include, e.g., allyl, 1-butenyl, 2-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The term “thioaryloxy” refers to an —S-aryl radical. The terms “aralkoxy” and “heteroaralkoxy” refer to an —O-aralkyl radical and —O-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O— heterocycloalkenyl radical, respectively. The term “heterocyclyloxy” refers to an —O— heterocyclyl radical. The terms “alkenyloxy” and “alkynyloxy” refer to —O-alkenyl and —O-alkynyl radicals, respectively.

The term “heterocyclyl” refers to a saturated monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). The heteroatom or ring carbon is the point of attachment of the heterocyclyl substituent to another moiety. Any atom can be optionally substituted, e.g., by one or more substituents. The heterocyclyl groups can contain fused rings. Fused rings are rings that share a common carbon atom. Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, or norbornenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). A ring carbon (e.g., saturated or unsaturated) or heteroatom is the point of attachment of the heterocycloalkenyl substituent. Any atom can be optionally substituted, e.g., by one or more substituents. The heterocycloalkenyl groups can contain fused rings. Fused rings are rings that share a common carbon atom. Heterocycloalkenyl groups can include, e.g., tetrahydropyridyl, and dihydropyranyl.

The term “aryl” refers to an aromatic monocyclic, bicyclic, or tricyclic hydrocarbon ring system, wherein any ring atom can be optionally substituted, e.g., by one or more substituents. Aryl groups can contain fused rings. Fused rings are rings that share a common carbon atom. Aryl moieties can include, e.g., phenyl, naphthyl, anthracenyl, and pyrenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). Any atom can be optionally substituted, e.g., by one or more substituents. Heteroaryl groups can contain fused rings. Fused rings are rings that share a common carbon atom. Heteroaryl groups include pyridyl, thienyl, furyl (furanyl), imidazolyl, isoquinolyl, quinolyl and pyrrolyl.

The term “oxo” refers to an oxygen atom, which forms a carbonyl (C═O) when attached to carbon, or which forms part of a sulfinyl or sulfonyl group when attached to a sulfur atom, or which forms part of an N-oxide when attached to a nitrogen. The term “thioxo” refers to an oxygen atom, which forms a thiocarbonyl (C═S) when attached to carbon.

The expression “optionally substituted” when used in conjunction with any structure described herein (e.g., alkyl, cycloalkyl, alkenyl, alkynyl, aralkyl, heteroaralkyl, heterocyclyl, heterocycloalkenyl, cycloalkenyl, aryl, heteroaryl) means that the referenced structure can either be unsubstituted or that any one or more (e.g., 1-10, 1, 2, 3, 4, or 5) hydrogen atoms (and/or halo atoms in the case of a haloalkyl) in the structure can be replaced by a substituent (i.e., a group other that hydrogen group that is attached to any atom of the aforementioned structures).

When R, R^{a}, R^{f}, R^{g}, R^{h}, Rⁱ, R^{j}, R³, R⁴, R^{3′}, R^{4′}, R^{N1}, or R^{N2} is an aryl or heteroaryl group (or a group that contains an aryl or heteroaryl group, e.g., an aryloxy group or heteroaryloxy group) that is substituted with one or more (e.g., 1-10, 1-5, 1-4, 1-3, 1-2, or 1) substituents, each of the substituents can be independently selected from (referred to collectively as “Group A”):

(i) halo; NR^{f}R^{g}; nitro; azido; hydroxy; C₁-C₁₂ (e.g., C₁-C₁₀, C₁-C₆, C₁-C₄, or C₁-C₃) alkoxy or C₁-C₁₂ (e.g., C₁-C₁₀, C₁-C₆, C₁-C₄, or C₁-C₃) thioalkoxy, each of which is optionally substituted with from 1-5 (e.g., 1-4, 1-3, 1-2, or 1) substituents independently selected from Group C below; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy, heteroaryloxy including 5-20 (e.g., 5-12, 5-10, or 5-6) atoms, or thioaryloxy including 5-20 (e.g., 5-12, 5-10, or 5-6) atoms, each of which is optionally substituted with from 1-10 (e.g., 1-5, 1-4, 1-3, 1-2, or 1) R^{a}; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy, C₃-C₁₆ (e.g., C₃-C₁₀, C₃-C₈, C₃-C₆) cycloalkenyloxy, heterocyclyloxy including 3-16 (e.g., 3-10, 3-8, 3-6) atoms, heterocycloalkenyloxy including 3-16 (e.g., 3-10, 3-8, 3-6) atoms, C₇-C₂₀ (e.g., C₇-C₁₆, C₇-C₁₂, C₇-C₁₀) aralkoxy, or heteroaralkoxy including 6-20 (e.g., 6-14, 6-10) atoms, each of which is optionally substituted with from 1-5 (e.g., 1-4, 1-3, 1-2, or 1) substituents independently selected from Group B below; mercapto; cyano; —C(O)R^{h}, —C(O)OR^{h}; —OC(O)R^{h}; —C(O)SR^{h}; —SC(O)R^{h}; —C(S)SR^{h}; —SC(S)R^{h}; —C(O)NR^{f}R^{g}; —NRⁱC(O)R^{j}; —OC(O)NR^{f}R^{g}; or 2 adjacent substituents on an aryl or heteroaryl ring (or a group that contains an aryl or heteroaryl group) together form C₁-C₃ alkylenedioxy;

(ii) C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl; each of which is optionally substituted with from 1-5 (e.g., 1-4, 1-3, 1-2, or 1) substituents independently selected from Group D below; or

(iii) C₇-C₂₀ (e.g., C₇-C₁₆, C₇-C₁₂, C₇-C₁₀) aralkyl; C₃-C₁₆ (e.g., C₃-C₁₀, C₃-C₈, C₃-C₆) cycloalkyl; heteroaralkyl including 6-20 (e.g., 6-14, 6-10) atoms; C₃-C₁₆ (e.g., C₃-C₁₀, C₃-C₈, C₃-C₆) cycloalkenyl; heterocyclyl including 3-16 (e.g., 3-10, 3-8, 3-6) atoms; or heterocycloalkenyl including 3-16 (e.g., 3-10, 3-8, 3-6) atoms; each of which is optionally substituted with from 1-5 (e.g., 1-4, 1-3, 1-2, or 1) substituents independently selected from Group B below; or

(iv) C₂-C₂₀ (e.g., C₂-C₁₂, C₂-C₁₀, C₂-C₆, or C₂-C₄) alkenyl or C₂-C₂₀ (e.g., C₂-C₁₂, C₂-C₁₀, C₂-C₆, or C₂-C₄) alkynyl; or

(v) C₆-C₁₆ (e.g., C₆-C₁₄, C₆-C₁₀, or phenyl) aryl or heteroaryl including 5-16 (e.g., 5-12, 5-10, or 5-6) atoms, each of which is optionally substituted with from 1-10 (e.g., 1-5, 1-4, 1-3, 1-2, or 1) R^{a′}.

R^{a′} at each occurrence is, independently, C₁-C₁₂ (e.g., C₁-C₁₀, C₁-C₆, C₁-C₄, or C₁-C₃) alkyl, C₁-C₁₂ (e.g., C₁-C₁₀, C₁-C₆, C₁-C₄, or C₁-C₃) haloalkyl, C₂-C₁₂ (e.g., C₂-C₁₀, C₂-C₆, or C₂-C₄) alkenyl; C₂-C₁₂ (e.g., C₂-C₁₀, C₂-C₆, or C₂-C₄) alkynyl; C₃-C₁₆ (e.g., C₃-C₁₀, C₃-C₈, C₃-C₆) cycloalkyl; C₃-C₁₆ (e.g., C₃-C₁₀, C₃-C₈, C₃-C₆) cycloalkenyl, heterocyclyl including 3-16 (e.g., 3-10, 3-8, 3-6) atoms, heterocycloalkenyl including 3-16 (e.g., 3-10, 3-8, 3-6) atoms; C₇-C₂₀ (e.g., C₇-C₁₆, C₇-C₁₂, C₇-C₁₀) aralkyl; C₆-C₁₆ (e.g., C₆-C₁₄, C₆-C₁₀, or phenyl) aryl; heteroaryl including 5-16 (e.g., 5-12, 5-10, or 5-6) atoms; halo; NR^{f}R^{g}; nitro; azido, hydroxy; C₁-C₁₂ (e.g., C₁-C₁₀, C₁-C₆, C₁-C₄, or C₁-C₃) alkoxy; C₁-C₁₂ (e.g., C₁-C₁₀, C₁-C₆, C₁-C₄, or C₁-C₃) thioalkoxy; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy; heteroaryloxy including 5-20 (e.g., 5-12, 5-10, or 5-6) atoms; thioaryloxy including 5-20 (e.g., 5-12, 5-10, or 5-6) atoms; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy; C₃-C₁₆ (e.g., C₃-C₁₀, C₃-C₈, C₃-C₆) cycloalkenyloxy; heterocyclyloxy including 3-16 (e.g., 3-10, 3-8, 3-6) atoms; heterocycloalkenyloxy including 3-16 (e.g., 3-10, 3-8, 3-6) atoms; C₇-C₂₀ (e.g., C₇-C₁₆, C₇-C₁₂, C₇-C₁₀) aralkoxy; heteroaralkoxy including 6-20 (e.g., 6-14, 6-10) atoms; mercapto; cyano; —C(O)R^{h}, —C(O)OR^{h}; —OC(O)R^{h}; —C(O)SR^{h}; —SC(O)R^{h}; —C(S)SR^{h}; —SC(S)R^{h}; —C(O)NR^{f}R^{g}; —NRⁱC(O)R^{j}; —OC(O)NR^{f}R^{g}; or 2 adjacent substituents on an aryl or heteroaryl ring (or a group that contains an aryl or heteroaryl group) together form C₁-C₃ alkylenedioxy.

When R, R^{a}, R^{f}, R^{g}, R^{h}, Rⁱ, R^{j}, R³, R⁴, R^{3′}, R^{4′}, R^{N1} or R^{N2} is an aralkyl, cycloalkyl; heteroaralkyl, cycloalkenyl, heterocyclyl, or heterocycloalkenyl group (or a group that contains an aryl or heteroaryl group, e.g., a cycloalkyloxy, cycloalkenyloxy; heterocyclyloxy, heterocycloalkenyloxy, aralkoxy; or heteroaralkoxy) that is substituted with one or more (e.g., 1-10, 1-5, 1-4, 1-3, 1-2, or 1) substituents, each of the substituents can be independently selected from (referred to collectively as “Group B”):

(i) halo; NR^{f}R^{g}; nitro; azido; hydroxy; oxo, thioxo, ═NR^{k}, C₁-C₁₂ (e.g., C₁-C₁₀, C₁-C₆, C₁-C₄, or C₁-C₃) alkoxy or C₁-C₁₂ (e.g., C₁-C₁₀, C₁-C₆, C₁-C₄, or C₁-C₃) thioalkoxy, each of which is optionally substituted with from 1-5 (e.g., 1-4, 1-3, 1-2, or 1) substituents independently selected from Group C below; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy, heteroaryloxy including 5-20 (e.g., 5-12, 5-10, or 5-6) atoms, or thioaryloxy including 5-20 (e.g., 5-12, 5-10, or 5-6) atoms, each of which is optionally substituted with from 1-5 (e.g., 1-4, 1-3, 1-2, or 1) substituents independently selected from Group A above; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy, C₃-C₁₆ (e.g., C₃-C₁₀, C₃-C₈, C₃-C₆) cycloalkenyloxy, heterocyclyloxy including 3-16 (e.g., 3-10, 3-8, 3-6) atoms, heterocycloalkenyloxy including 3-16 (e.g., 3-10, 3-8, 3-6) atoms, C₇-C₂₀ (e.g., C₇-C₁₆, C₇-C₁₂, C₇-C₁₀) aralkoxy, or heteroaralkoxy including 6-20 (e.g., 6-14, 6-10) atoms, each of which is optionally substituted with 1-5 (e.g., 1-4, 1-3, 1-2, or 1) R^{b′}; mercapto; cyano; —C(O)R^{h}, —C(O)OR^{h}; —OC(O(O)R^{h}; —C(O)SR^{h}; —SC(O)R^{h}; C(S)SR^{h}; —SC(S)R^{h}; —C(O)NR^{f}R^{g}; —NRⁱC(O)R^{j}; —OC(O)NR^{f}R^{g}; or

(ii) C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl; each of which is optionally substituted with from 1-5 (e.g., 1-4, 1-3, 1-2, or 1) substituents independently selected from Group D below; or

(iii) C₇-C₂₀ (e.g., C₇-C₁₆, C₇-C₁₂, C₇-C₁₀) aralkyl; C₃-C₁₆ (e.g., C₃-C₁₀, C₃-C₈, C₃-C₆) cycloalkyl; heteroaralkyl including 6-20 (e.g., 6-14, 6-10) atoms; C₃-C₁₆ (e.g., C₃-C₁₀, C₃-C₈, C₃-C₆) cycloalkenyl; heterocyclyl including 3-16 (e.g., 3-10, 3-8, 3-6) atoms; or heterocycloalkenyl including 3-16 (e.g., 3-10, 3-8, 3-6) atoms; each of which is optionally substituted with from 1-10 (e.g., 1-5, 1-4, 1-3, 1-2, or 1) R^{b′}; or

(iv) C₂-C₂₀ (e.g., C₂-C₁₂, C₂-C₁₀, C₂-C₆, or C₂-C₄) alkenyl or C₂-C₂₀ (e.g., C₂-C₁₂, C₂-C₁₀, C₂-C₆, or C₂-C₄) alkynyl; or

(v) C₆-C₁₆ (e.g., C₆-C₁₄, C₆-C₁₀, or phenyl) aryl or heteroaryl including 5-16 (e.g., 5-12, 5-10, or 5-6) atoms, each of which is optionally substituted with from 1-5 (e.g., 1-4, 1-3, 1-2, or 1) substituents independently selected from Group A above.

R^{b′} at each occurrence is, independently, C₁-C₁₂ (e.g., C₁-C₁₀, C₁-C₆, C₁-C₄, or C₁-C₃) alkyl or C₁-C₁₂ (e.g., C₁-C₁₀, C₁-C₆, C₁-C₄, or C₁-C₃) haloalkyl, each of which is optionally substituted with from 1-5 (e.g., 1-4, 1-3, 1-2, or 1) substituents independently selected from Group D below; C₂-C₁₂ (e.g., C₂-C₁₀, C₂-C₆, or C₂-C₄) alkenyl; C₂-C₁₂ (e.g., C₂-C₁₀, C₂-C₆, or C₂-C₄) alkynyl; C₃-C₁₆ (e.g., C₃-C₁₀, C₃-C₈, C₃-C₆) cycloalkyl; C₃-C₁₆ (e.g., C₃-C₁₀, C₃-C₈, C₃-C₆) cycloalkenyl, heterocyclyl including 3-16 (e.g., 3-10, 3-8, 3-6) atoms, heterocycloalkenyl including 3-16 (e.g., 3-10, 3-8, 3-6) atoms; C₇-C₂₀ (e.g., C₇-C₁₆, C₇-C₁₂, C₇-C₁₀) aralkyl; C₆-C₁₆ (e.g., C₆-C₁₄, C₆-C₁₀, or phenyl) aryl; heteroaryl including 5-16 (e.g., 5-12, 5-10, or 5-6) atoms; halo; NR^{f}R^{g}; nitro; azido, hydroxy; oxo, thioxo, ═NR^{k}, C₁-C₁₂ (e.g., C₁-C₁₀, C₁-C₆, C₁-C₄, or C₁-C₃) alkoxy; C₁-C₁₂ (e.g., C₁-C₁₀, C₁-C₆, C₁-C₄, or C₁-C₃) thioalkoxy; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy; heteroaryloxy including 5-20 (e.g., 5-12, 5-10, or 5-6) atoms; thioaryloxy including 5-20 (e.g., 5-12, 5-10, or 5-6) atoms; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy; C₃-C₁₆ (e.g., C₃-C₁₀, C₃-C₈, C₃-C₆) cycloalkenyloxy; heterocyclyloxy including 3-16 (e.g., 3-10, 3-8, 3-6) atoms; heterocycloalkenyloxy including 3-16 (e.g., 3-10, 3-8, 3-6) atoms; C₇-C₂₀ (e.g., C₇-C₁₆, C₇-C₁₂, C₇-C₁₀) aralkoxy; heteroaralkoxy including 6-20 (e.g., 6-14, 6-10) atoms; mercapto; cyano; —C(O)R^{h}, —C(O)OR^{h}; —OC(O)R^{h}; —C(O)SR^{h}; —SC(O)R^{h}; —C(S)SR^{h}; —SC(S)R^{h}; —C(O)NR^{f}R^{g}; —NRⁱC(O)R^{j}; or —OC(O)NR^{f}R^{g}.

When R, R^{a}, R^{f}, R^{g}, R^{h}, Rⁱ, R^{j}, R³, R⁴, R^{3′}, R^{4′}, R^{N1}, or R^{N2} is an alkoxy or thioalkoxy group that is substituted with one or more (e.g., 1-10, 1-5, 1-4, 1-3, 1-2, or 1) substituents, each of the substituents can be independently selected from (referred to collectively as “Group C”): NR^{f}R^{g}; nitro; azido; hydroxy; oxo, thioxo, ═NR^{k}, C₁-C₁₂ (e.g., C₁-C₁₀, C₁-C₆, C₁-C₄, or C₁-C₃) alkoxy or C₁-C₁₂ (e.g., C₁-C₁₀, C₁-C₆, C₁-C₄, or C₁-C₃) thioalkoxy; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy, heteroaryloxy including 5-20 (e.g., 5-12, 5-10, or 5-6) atoms, or thioaryloxy including 5-20 (e.g., 5-12, 5-10, or 5-6) atoms, each of which is optionally substituted with from 1-5 (e.g., 1-4, 1-3, 1-2, or 1) substituents independently selected from Group A above; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy, C₃-C₁₆ (e.g., C₃-C₁₀, C₃-C₈, C₃-C₆) cycloalkenyloxy, heterocyclyloxy including 3-16 (e.g., 3-10, 3-8, 3-6) atoms, heterocycloalkenyloxy including 3-16 (e.g., 3-10, 3-8, 3-6) atoms, C₇-C₂₀ (e.g., C₇-C₁₆, C₇-C₁₂, C₇-C₁₀) aralkoxy, or heteroaralkoxy including 6-20 (e.g., 6-14, 6-10) atoms, each of which is optionally substituted; mercapto; cyano; —C(O)R^{h}, —OC(O)R^{h}; —C(O)SR^{h}; —SC(O)R^{h}; —C(S)SR^{h}; —SC(S)R^{h}; —C(O)NR^{f}R^{g}′; —NRⁱC(O)R^{j}; or —OC(O)NR^{f}R^{g}.

When R^{F1}, R^{F2}, R^{F1′}, R^{F2′}, R, R^{a}, R^{f}, R^{g}, R^{h}, Rⁱ, R³, R⁴, R^{3′}, R^{4′}, R^{N1} or R^{N2} is an alky or haloalkyl group (including a fluoroalkyl group) that is substituted with one or more (e.g., 1-10, 1-5, 1-4, 1-3, 1-2, or 1) substituents, each of the substituents can be independently selected from (referred to collectively as “Group D”): NR^{f}R^{g}; nitro; azido; hydroxy; oxo; thioxo; ═NR^{k}; C₁-C₁₂ (e.g., C₁-C₁₀, C₁-C₆, C₁-C₄, or C₁-C₃) alkoxy or C₁-C₁₂ (e.g., C₁-C₁₀, C₁-C₆, C₁-C₄, or C₁-C₃) thioalkoxy, each of which is optionally substituted with from 1-5 (e.g., 1-4, 1-3, 1-2, or 1) substituents independently selected from Group C above; C₁-C₁₂ (e.g., C₁-C₁₀, C₁-C₆, C₁-C₄, or C₁-C₃) haloalkoxy; C₆-C₁₆ (e.g., C₆-C₁₄, C₆-C₁₀, or phenyl) aryloxy, C₆-C₁₆ (e.g., C₆-C₁₄, C₆-C₁₀, or phenyl) thioaryloxy, heteroaryloxy including 5-20 (e.g., 5-12, 5-10, or 5-6) atoms, or thioaryloxy including 5-20 (e.g., 5-12, 5-10, or 5-6) atoms, each of which is optionally substituted with from 1-5 (e.g., 1-4, 1-3, 1-2, or 1) substituents independently selected from Group A above; C₂-C₁₂ (e.g., C₂-C₁₀, C₂-C₆, or C₂-C₄) alkenyloxy; C₂-C₁₂ (e.g., C₂-C₁₀, C₂-C₆, or C₂-C₄) alkynyloxy; C₃-C₁₆ (e.g., C₃-C₁₀, C₃-C₈, C₃-C₆) cycloalkyloxy, C₃-C₁₆ (e.g., C₃-C₁₀, C₃-C₈, C₃-C₆) cycloalkenyloxy, heterocyclyloxy including 3-16 (e.g., 3-10, 3-8, 3-6) atoms, heterocycloalkenyloxy including 3-16 (e.g., 3-10, 3-8, 3-6) atoms, C₇-C₂₀ (e.g., C₇-C₁₆, C₇-C₁₂, C₇-C₁₀) aralkoxy, or heteroaralkoxy including 6-20 (e.g., 6-14, 6-10) atoms, each of which is optionally substituted with from 1-5 (e.g., 1-4, 1-3, 1-2, or 1) substituents independently selected from Group A above; mercapto; cyano; —C(O)R^{h}, —C(O)OR^{h}; —OC(O)R^{h}; —C(O)SR^{h}; —SC(O)R^{h}; —C(S)SR^{h}; —SC(S)R^{h}; —C(O)NR^{f}R^{g}; —NRⁱC(O)R^{j}; or —OC(O)NR^{f}R^{g}.

In embodiments, Groups C and D can further include C₃-C₁₀ (e.g., C₃-C₆) cycloalkyl and heterocyclyl including 3-8 (e.g., 3-6 or 5-6) atoms, each of which can be optionally substituted with from 1-5 (e.g., 1-4, 1-3, 1-2, or 1) substituents independently selected from Group A above.

R^{k} can be as defined for R^{f}, R^{g}, and R^{h}.

When R, R^{a}, R^{f}, R^{g}, R^{h}, Rⁱ, R^{j}, R³, R⁴, R^{3′}, R^{4′}, R^{N1} or R^{N2} is an alkenyl or alkynyl group that is substituted with one or more (e.g., 1-10, 1-5, 1-4, 1-3, 1-2, or 1) substituents, each of the substituents can be independently selected from halo or a Group C or D substituent.

The details of one or more embodiments of the invention are set forth in the description below. Other features and advantages of the invention are in the claims.

### DETAILED DESCRIPTION

In general, the starting material can be any organic compound that is substituted with one or more nitrile groups (see, e.g., the Summary section of the specification). In some embodiments, starting materials (as well as intermediates and products formed in the methods described herein) can also include compounds described generically, subgenerically, and specifically in US 2007-0219198, filed on Feb. 7, 2007, which is incorporated herein by reference in its entirety. The inventors named on the present application and on US 2007-0219198 are obligated to assign to the same assignee. Starting materials (as well as intermediates and products formed in the methods described herein) can also include one or more structural features described in US 2007-0219198.

In some embodiments, the nitrile can be attached to an optionally substituted C₆-C₁₀ aryl (e.g., phenyl). In other embodiments, the nitrile can be attached to an optionally substituted C₇-C₁₂ aralkyl, e.g., benzyl.

In some embodiments, the starting material can include a substituent having a formula —C(O)R^{e}, wherein R^{e} is C₁-C₆ alkyl (e.g., R^{e} can be CH₃). In other embodiments, substituent selection for the starting materials can be made on the basis of whether a particular substituent is known to be stable or inert to a particular fluoroalkylating agent or classes thereof (e.g., the fluoroalkylating agents described herein). Thus, in some embodiments, the starting material can be only further substituted (i.e., in addition to the nitrile) with moieties known to be stable or inert to a particular fluoroalkylating agent or classes thereof (e.g., the fluoroalkylating agents described herein).

The methods described herein also extend to the use of starting materials and intermediates having masked nitrile groups or other substituents, which can provide a nitrile group (or its equivalent) in situ (e.g., in situ in the presence of the fluoroalkylating agent).

In some embodiments, the fluoroalkylating agent can be a perfluoroalkylating agent (e.g., a trifluoromethylating agent).

In some embodiments, the fluoroalkylating agent can be a nucleophilic fluoroalkylating agent (e.g., a fluoroalkylating agent that can undergo 1, 2 addition to an enolizable or non-enolizable carbonyl compound).

In some embodiments, the fluoroalkylating agent can be a silicon-based reagent, e.g., a compound having formula (III):

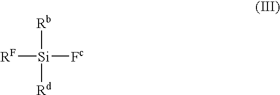

in which:

R^{F} can be C₁-C₆ fluoroalkyl; and

each of R^{b}, R^{c}, and R^{d} can be, independently, C₁-C₁₂ alkyl or C₂-C₁₂ alkenyl, each of which is optionally substituted.

In embodiments, each of R^{b}, R^{c}, and R^{d} can be, independently, C₁-C₄ alkyl (e.g., CH₃ or CH₂CH₃). In other embodiments, one of R^{b}, R^{c}, and R^{d} is C₂-C₄ alkenyl (e.g., CH═CH₂), and the other two are each, independently, C₁-C₄ alkyl (e.g., CH₃ or CH₂CH₃).

In embodiments, R^{F} can be C₁-C₄ perfluoroalkyl (e.g., CF₃).

An exemplary fluoroalkylating agent of formula (III) is CF₃Si(CH₃)₃, sometimes referred to as Ruppert's reagent or the Ruppert-Prakash reagent. Methods for the synthesis and use of Ruppert's reagent are described in, e.g., Prakash, G. K. S.; Krishnamurti, R.; Olah, G. A. J. Am. Chem. Soc. 1989, 111, 393; Prakash, G. K. S.; Yudin, A. K. Chem. Rev. 1997, 97, 757; and Prakash, G. K. S.; Hu, J.; Olah, G. A., J. Org. Chem. 2003, 68, 4457, incorporated herein by reference thereto.

Other fluoroalkylating agent of formula (III) include, without limitation, triethyltrifluoromethylsilane, CF₃Si(CH₂CH₃)₃, see, e.g., U.S. Pat. No. 5,008,425; and vinyl(trifluoromethyl)dimethylsilane, which is commercially available, e.g., from the following vendors: ABCR GmbH & CO. (Ryan Scientific in the US), Oakwood Products, Inc. (US), and Gelest, Inc. (US).

In some embodiments, about 2 equivalents (or a relatively small excess thereof) of the fluoroalkylating agent of formula (III) is used per nitrile functional group.

Typically, a moiety having a relatively strong affinity for silicon (e.g., a fluoride ion source or oxygen nucleophile) is present during the reaction between the nitrile-containing compound and the compound of formula (III). In certain embodiments, about 1 equivalent of fluoride ion is used per equivalent of nitrile-containing compound.

In some embodiments, the fluoroalkylating agent can be a fluoroalkyl-containing salt or ionic complex, e.g., an ionic complex formed upon interaction of a fluoroalkyl halide (e.g., a fluoroalkyl iodide) and a reducing agent. For example, trifluoromethyl iodide (CF₃I) can be used as a nucleophilic trifluoromethylating agent under the activation of electron-donating tetrakis-(dimethylamino)ethylene (TDAE). See, e.g., Ait-Mohand, S.; Takechi, N.; Medebielle, M.; Dolbier, W. Jr. Org. Lett. 2001, 3, 4271. As a further example, see J. Org. Chem. 2006, 71, 3564, which describes the use of other fluoroalkyl iodides to introduce other perfluoroalkyl groups (R_{F}), such as C₂F₅ or n-C₄F₉, by using R_{F}I and TDAE.

In these embodiments, the methods can further include reacting a compound having formula (IV): R^{F}—X, wherein R^{F} is C₁-C₆ fluoroalkyl; and X is halo; with a reducing agent (e.g., TDAE). In embodiments, X can be iodo. In embodiments, R^{F} is CF₃, CF₂CF₃, or (CF₂)₃CF₃.

In some embodiments, the fluoroalkylating agent can be a hemiaminal that is formed between fluoral (CF₃CHO) and a cyclic amine. For example, the fluoroalkylating agent can be compound having formula (V):

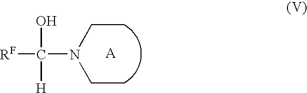

in which R^{F} can be C₁-C₆ fluoroalkyl; and ring A is optionally substituted morpholinyl or piperazinyl.

In embodiments, R^{F} can be CF₃. See, e.g., Billard, T. B.; Langlois, B. R. Org. Lett. 2000, 2, 2101; Billard, T.; Langlois, B. R.; Blond, G. Eur. J. Org. Chem. 2001, 1467; Billard, T.; Langlois, B. R. J. Org. Chem. 2002, 67, 997; and Langlois, B. R.; Billard, T. Synthesis 2003, 185.

In these embodiments, a base is typically present during the reacting of the compound of the nitrile-containing compound and the compound of formula (V). By way of example, the base can be a metal salt (e.g., K⁺) of a C₁-C₆ alkoxide (e.g., tert-butoxide).

In some embodiments, the fluoroalkylating agent can be a compound having formula (VI): Ar—S(O)ₓ—R^{F}; in which Ar can be optionally substituted phenyl; x can be 0, 1 or 2 (e.g., 1 or 2); and R^{F} is C₁-C₆ fluoroalkyl.

In embodiments, R^{F} can be CF₃. In embodiments, x can be 2. See, e.g., U.S. Pat. No. 7,087,789 and Prakash, G. K. S.; Hu, J.; Olah, G. A., J. Org. Chem. 2003, 68, 4457.

In these embodiments, a base is typically present during the reacting of the compound of the nitrile-containing compound and the compound of formula (VI). By way of example, the base can be a metal salt (e.g., K⁺) of a C₁-C₆ alkoxide (e.g., tert-butoxide).

In some embodiments, the fluoroalkylating agent can be fluoroform (CF₃H). Methods for the synthesis, deprotonation, and trifluoromethylation of fluoroform are described in, e.g., Webster J. L.; Lerou, J. J. U.S. Pat. No. 5,446,218, 1995; Shono, T.; Ishifume, M.; Okada, T.; Kashimura, S. J. Org. Chem. 1991, 56, 2; Barhdadi, R.; Troupel, M.; Perichon, J. Chem. Comm. 1998, 1251; Folleas, B.; Marek, I.; Normant, J.-F.; Saint-Jalmes, L. Tetrahedron Lett. 1998, 39, 2973; Folleas, B.; Marek, I.; Normant, J.-F.; Saint-Jalmes, L. Tetrahedron 2000, 56, 275; Russell, J.; Roques, N. Tetrahedron 1998, 54, 13771; Large, S.; Roques, N.; Langlois, B. R J. Org. Chem. 2000, 65, 8848; Roques, N.; Russell, J.; Langlois, B.; Saint-Jalmes, L.; Large, S. PCT Int. Appl. 1998, WO 9822435; and Roques, N.; Mispelaere, C. Tetrahedron Lett. 1999, 40, 6411.

Other fluoroalkylating agents include trifluoromethylcopper reagents; sodium trifluoroacetate used in conjunction with copper halide catalysts, see, e.g., Tet. Lett. 2005, 46, 3161); trifluoroacetic and trifluoromethanesulfinic acid derivatives; trifluoroacetamides, trifluoroacetophenone and adducts thereof, and trifluoromethanesulfinamides. See, e.g., Angew. Chem. Int. Ed. 2003, 42, 3133; Synlett. 2004, 2119; and Chem. Eur. J. 2005, 11, 939; Langlois, B. R.; Billard, T. Synthesis 2003, 185; Jablonski, L.; Joubert, J.; Billard, T.; Langlois, B. R. Synlett 2003, 230; Inschauspe, D.; Sortais, J.-P.; Billard, T.; Langlois, B. R. Synlett 2003, 233; and Jablonski, L.; Billard, T.; Langlois, B. R. Tetrahedron Lett. 2003, 44, 1055; and Synlett 2002, 646.

In some embodiments, the fluoroalkylating agent can be agent that can used to introduce a difluoromethyl group (—CF₂H).

In certain embodiments, the fluoroalkylating agent can be difluoromethyl phenyl sulfone (PhSO₂CF₂H). See, e.g., Eur. J. Org. Chem. 2005, 2218; Org. Lett. 2004, 6, 4315; U.S. Pat. No. 7,087,789; and Angew. Chem. Int. Ed. 2005, 44, 5882.

In certain embodiments, the fluoroalkylating agent can be TMS—CF₂SO₂Ph. See, e.g., Tet. Lett. 2005, 46, 8273.

In certain embodiments, the fluoroalkylating agent can be TMS—CF₂H, TMS—CF₂SePh, TMSCF₂TMS, or TMS—SiCF₂SPh. See, e.g., Yudin, A. K.; Prakash, G. K. S.; Deffieux, D.; Bradley, M.; Bau, R.; Olah, G. A. J. Am. Chem. Soc. 1997, 119, 1572 1581.

In some embodiments, the method can further include other protecting group and/or functional group manipulation steps. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, starting materials and reagents can be synthesized according to methods described herein and/or conventional, organic chemical synthesis methods from commercially available starting materials and reagents. As can be appreciated by the skilled artisan, further methods of synthesizing such compounds will be evident to those of ordinary skill in the art.

The reaction products and intermediates described herein can be separated from a reaction mixture and further purified by a method such as column chromatography, high-pressure liquid chromatography, or recrystallization.

The starting materials, intermediates, and products of the methods described herein may contain two or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented. All such isomeric forms of such compounds are expressly included in the present invention. All crystal forms of the compounds described herein are expressly included in the present invention.

The compounds of this invention include the compounds themselves, as well as their salts and their S or N-oxides, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The invention will be further described in the following examples. It should be understood that these examples are for illustrative purposes only and are not to be construed as limiting this invention in any manner.

### EXAMPLES

### Example 1

### 2,2,2-Trifluoro-1-[3-(piperidine-1-sulfonyl)-phenyl]-1-trifluoromethyl-ethylamine

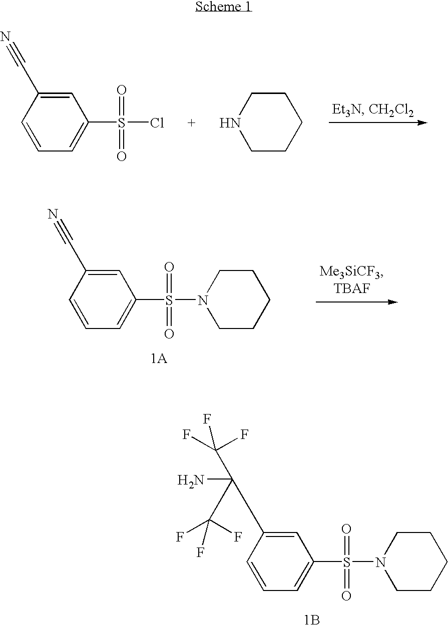

Step A. To a mixture of 3-cyano-benzenesulfonyl chloride (170 mg, 0.84 mmol, 1A) and triethylamine (0.24 mL, 1.69 mmol) in 2 mL dichloromethane at 0° C. was added piperidine (124 μL, 1.26 mmol). The reaction mixture was stirred at 25° C. for 16 h and concentrated to give a yellow oily residue. Flush column chromatography of the residue (silica gel, hexane:ethyl acetate=1:3) provided compound 3-(piperidine-1-sulfonyl)-benzonitrile (189 mg, 90%) as a white solid.

¹H NMR (400 MHz, CDCl₃): δ 1.42-1.52 (m, 2H), 1.62-1.72 (m, 4H), 2.99-3.08 (m, 4H), 7.71 (dd, J=7.83, 7.83 Hz, 1H), 7.89 (d, J=7.83 Hz, 1H), 8.00 (d, J=7.83 Hz, 1H), 8.07 (s, 1H).

Step B. To a mixture of 3-(piperidine-1-sulfonyl)-benzonitrile (80 mg, 0.32 mmol) and (trifluoromethyl)trimethylsilane (0.14 mL, 0.96 mmol) in 3 mL dry THF at 0° C. under nitrogen was added tetrabutylammonium fluoride (176 mg, 0.67 mmol) in 1 mL dry THF. The reaction mixture was stirred at 0° C. for 2 h and concentrated to give a yellow oily residue. Flush column chromatography of the residue (silica gel, hexane ethyl acetate=6:1) afforded 2,2,2-trifluoro-1-[3-(piperidine-1-sulfonyl)-phenyl]-1-trifluoromethyl-ethylamine 1B (55 mg, 55%) as a colorless oil and starting material 1A (28 mg, 0.112 mmol, 35%).

¹H NMR (400 MHz, CDCl₃): δ 1.37-1.50 (m, 2H), 1.56-1.70 (m, 4H), 2.24 (s, 2H), 2.92-3.09 (m, 4H), 7.63 (dd, J=8.08, 8.08 Hz, 1H), 7.87 (d, J=8.08 Hz, 1H), 8.01 (d, J=8.08 Hz, 1H), 8.20 (s, 1H).

### Example 2

### 2,2,2-Trifluoro-1-[4-(piperidine-1-sulfonyl)-phenyl]-1-trifluoromethyl-ethylamine

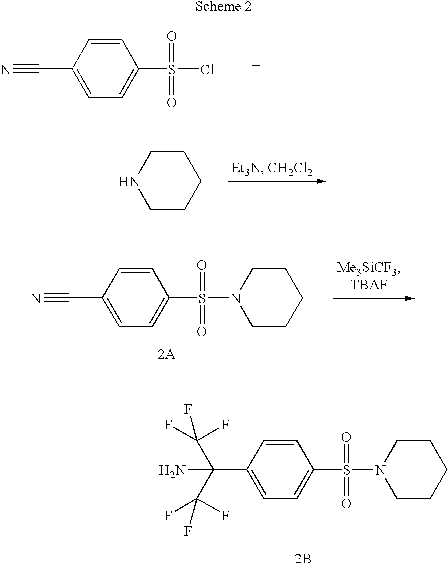

Step A. 4-(Piperidine-1-sulfonyl)-benzonitrile was prepared according to a procedure similar to that described in Example 1, Step A. 4-Cyano-benzenesulfonyl chloride (170 mg, 0.84 mmol) was converted to the desired product (200 mg, 95%) as a white solid.

¹H NMR (400 MHz, CDCl₃): δ 1.42-1.50 (m, 2H), 1.61-1.70 (m, 4H), 3.00-3.07 (m, 4H), 7.83 (d, J=8.84 Hz, 2H), 7.87 (d, J=8.84 Hz, 2H).

Step B. The title compound of Example 2 was prepared according to a procedure similar to that described in Example 1, Step B. 4-(Piperidine-1-sulfonyl)-benzonitrile (0.12 g, 0.48 mmol) was converted to the desired product (93.0 mg, 50%) as a colorless oil.

¹H NMR (400 MHz, CDCl₃): δ 1.40-1.50 (m, 2H), 1.62-1.71 (m, 4H), 2.23 (s, 2H), 2.99-3.09 (m, 4H), 7.82 (d, J=8.84 Hz, 2H), 7.97 (d, J=8.84 Hz, 2H).

### Example 3

### 2,2,2-Trifluoro-1-[3-(pyrrolidine-1-sulfonyl)-phenyl]-1-trifluoromethyl-ethylamine

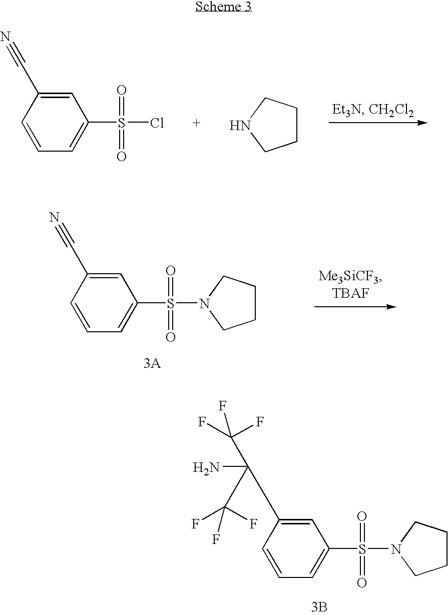

Step A. 3-(Pyrrolidine-1-sulfonyl)-benzonitrile was prepared according to a procedure similar to that described in Example 1, Step A. 3-Cyano-benzenesulfonyl chloride (200 mg, 1.00 mmol) was converted to the desired product (217 mg, 92%) as a white solid.

¹H NMR (400 MHz, CDCl₃): δ 1.79-1.85 (m, 4H), 3.24-3.32 (m, 4H), 7.69 (dd, J=7.83, 7.83 Hz, 1H), 7.87 (d, J=7.83 Hz, 1H), 8.07 (d, J=7.83 Hz, 1H), 8.13 (s, 1H).

Step B. The title compound of Example 3 was prepared according to a procedure similar to that described in Example 1, Step B. 3-(Pyrrolidine-1-sulfonyl)-benzonitrile 3A (76 mg, 0.32 mmol) was converted to the desired product (35.0 mg, 30%) as a colorless oil.

¹H NMR (400 MHz, CDCl₃): δ 1.73-1.80 (m, 4H), 2.23 (s, 2H), 3.22-3.29 (m, 4H), 7.64 (dd, J=7.83, 7.83 Hz, 1H), 7.94 (d, J=7.83 Hz, 1H), 8.02 (d, J=7.83 Hz, 1H), 8.28 (s, 1H).

### Example 4

### 3-(1-Amino-2,2,2-trifluoro-1-trifluoromethyl-ethyl)-N,N-diethyl-benzenesulfonamide

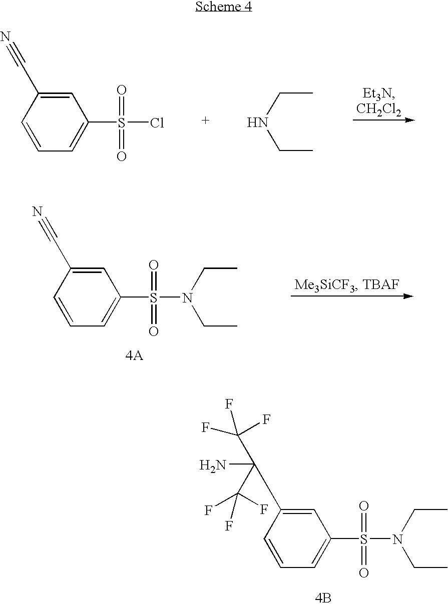

Step A. 3-Cyano-N,N-diethyl-benzenesulfonamide was prepared according to a procedure similar to that described in Example 1, Step A. 3-Cyano-benzenesulfonyl chloride (220 mg, 1.09 mmol) was converted to the desired product (250 mg, 96%) as a white solid.

¹H NMR (400 MHz, CDCl₃): δ 1.82 (t, J=6.82 Hz, 6H), 3.28 (q, J=6.82 Hz, 4H), 7.68 (dd, J=7.83, 7.83 Hz, 1H), 7.87 (d, J=7.83 Hz, 1H), 8.06 (d, J=7.83 Hz, 1H), 8.13 (s, 1H).

Step B. The title compound of Example 4 was prepared according to a procedure similar to that described in Example 1, Step B. 3-Cyano-N,N-diethyl-benzenesulfonamide 4A (98 mg, 0.41 mmol) was converted to the desired product (65.0 mg, 42%) as a colorless oil.

¹H NMR (400 MHz, CDCl₃): δ 1.11 (t, J=7.07 Hz, 6H), 2.24 (s, 2H), 3.26 (q, J=7.07 Hz, 4H), 7.60 (dd, J=7.83, 7.83 Hz, 1H), 7.92 (d, J=7.83 Hz, 1H), 7.97 (d, J=7.83 Hz, 1H), 8.26 (s, 1H).

### Example 5

### 3-(1-Amino-2,2,2-trifluoro-1-trifluoromethyl-ethyl)-N-(tert-butoxycarbonyl)-N-cyclohexyl-benzenesulfonamide

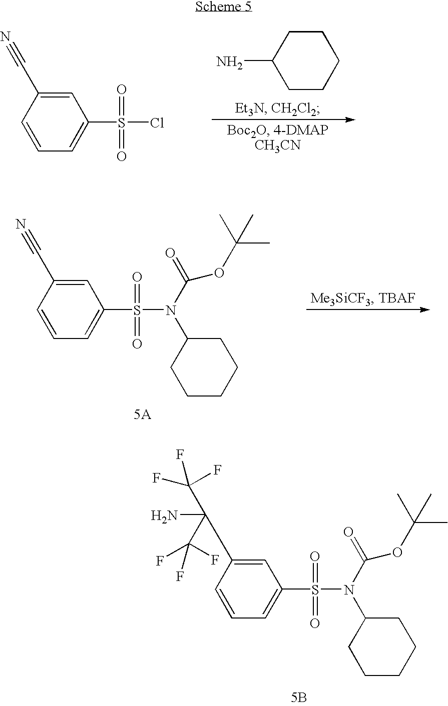

### Step A. 3-Cyano-N-(tert-butoxycarbonyl)-N-cyclohexyl-benzenesulfonamide

To a mixture of 3-cyano-benzenesulfonyl chloride (210 mg, 1.04 mmol) and (0.29 mL, 2.08 mmol) in 2 mL dichloromethane at 0° C. was added cyclohexylamine (180 μL, 1.56 mmol). The reaction mixture was stirred at 25° C. for 16 h and washed with water and brine. The organic layer was concentrated under rotary vacuum to give a yellow oily residue, which was then dried under high vacuum for 16 h to afford a yellow solid. To this yellow solid was added di-tert-butyl dicarbonate (330 mg, 1.52 mmol), 4-(dimethylamino)-pyridine (20 mg, 0.16 mmol), and 3 mL dry acetonitrile. The reaction mixture was stirred at 25° C. for 16 h and concentrated to give a yellow oily residue. Flush column chromatography of the residue (silica gel, hexane:ethyl acetate=5:1) provided 3-cyano-N-(tert-butoxycarbonyl)-N-cyclohexyl-benzenesulfonamide 5A (300 mg, 79%) as a white solid.

¹H NMR (400 MHz, CDCl₃): δ 1.09-1.24 (m, 2H), 1.29-1.47 (m, 2H), 1.38 (s, 9H), 1.78-1.92 (m, 4H), 2.10-2.24 (m, 2H), 4.25-4.37 (m, 1H), 7.66 (dd, J=7.83, 7.83 Hz, 1H), 7.87 (d, J=7.83 Hz, 1H), 8.13 (d, J=7.83 Hz, 1H), 8.19 (s, 1H).

Step B. The title compound of Example 5 was prepared according to a procedure similar to that described in Example 1, Step B. 3-Cyano-N-(tert-butoxycarbonyl)-N-cyclohexyl-benzenesulfonamide 5A (150 mg, 0.41 mmol) was converted to the desired product (71.0 mg, 34%) as a colorless oil.

¹H NMR (400 MHz, CDCl₃): δ 1.30 (s, 9H), 1.59-1.70 (m, 2H), 1.79-1.90 (m, 4H), 2.13-2.22 (m, 4H), 2.22 (s, 2H), 4.26-4.38 (m, 1H), 7.61 (dd, J=7.83, 7.83 Hz, 1H), 8.00 (d, J=7.83 Hz, 1H), 8.04 (d, J=7.83 Hz, 1H), 8.30-8.34 (s, 1H).

### Example 6

### 3-(1-Amino-2,2,2-trifluoro-1-trifluoromethyl-ethyl)-N-benzyl-N-(tert-butoxycarbonyl)-benzenesulfonamide

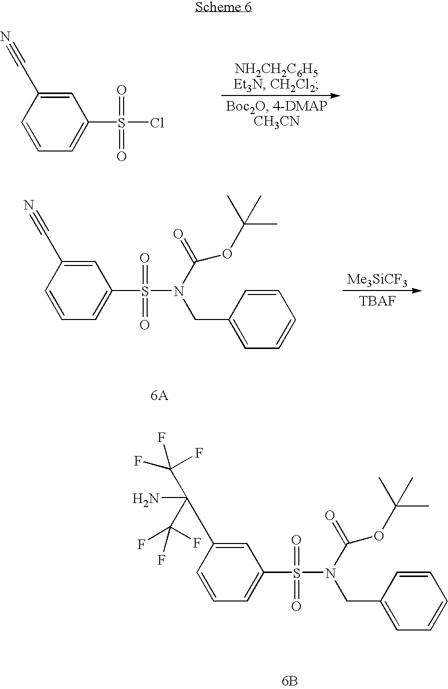

### Step A. N-Benzyl-N-(tert-butoxycarbonyl)-3-cyano-benzenesulfonamide

The title compound was prepared according to a procedure similar to that described in Example 5, Step A. 3-Cyano-benzenesulfonyl chloride (245 mg, 1.20 mmol) was converted to the desired product (388.7 mg, 87%) as a white solid.

¹H NMR (400 MHz, CDCl₃): δ 1.38 (s, 9H), 5.06 (s, 2H), 7.37-7.38 (m, 5H), 7.52 (dd, J=7.58, 7.58 Hz, 1H), 7.74 (s, 1H), 7.80 (d, J=7.58 Hz, 1H), 7.85 (d, J=7.58 Hz, 1H).

Step B. The title compound of Example 6 was prepared according to a procedure similar to that described in Example 1, Step B. N-Benzyl-N-(tert-butoxycarbonyl)-3-cyano-benzenesulfonamide 6A (165 mg, 0.44 mmol) was converted to the desired product (112.0 mg, 50%) as a colorless oil.

¹H NMR (400 MHz, CDCl₃): δ 1.29 (s, 9H), 2.02 (s, 2H), 5.07 (s, 2H), 7.28-7.43 (m, 5H), 7.52 (dd, J=8.34, 7.83 Hz, 1H), 7.85 (d, J=7.83 Hz, 1H), 7.95 (d, J=8.34 Hz, 1H), 8.04-8.10 (s, 1H).

### Example 7

### [3-(1-Amino-2,2,2-trifluoro-1-trifluoromethyl-ethyl)-phenyl]-piperidin-1-yl-methanone

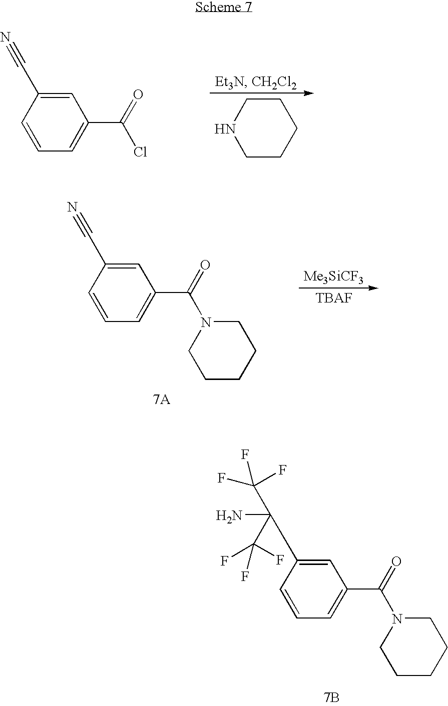

### Step A. 3-(Piperidine-1-carbonyl)-benzonitrile

The title compound was prepared according to a procedure similar to that described in Example 1, Step A. 3-Cyano-benzoyl chloride (150 mg, 0.91 mmol) was converted to the desired product (186.7 mg, 97%) as a mixture of two isomers in a 1:1 ratio. White solid.

¹H NMR (400 MHz, CDCl₃): δ 1.50-1.59 (m, 2H), 1.64-1.75 (m, 4H), 3.26-3.36 (m, 2H), 3.65-3.76 (m, 2H), 7.54 (dd, J=7.83, 7.83 Hz, 1H), 7.63 (d, J=7.83 Hz, 1H), 7.68 (s, 1H), 7.70 (d, J=7.83 Hz, 1H).

Step B. The title compound of Example 7 was prepared according to a procedure similar to that described in Example 1, Step B. 3-(Piperidine-1-carbonyl)-benzonitrile 7A (110 mg, 0.51 mmol) was converted to the desired product (80.0 mg, 44%) as a mixture of two isomers in a 1:1 ratio. White solid.

¹H NMR (400 MHz, CDCl₃): δ 1.48-1.57 (m, 2H), 1.63-1.74 (m, 4H), 2.20 (s, 2H), 3.23-3.36 (m, 2H), 3.64-3.79 (m, 2H), 7.46-7.54 (m, 2H), 7.78-7.84 (m, 2H).

### Example 8

### 3-(1-Amino-2,2,2-trifluoro-1-trifluoromethyl-ethyl)-N-(tert-butoxycarbonyl)-N-(4-methoxy-phenyl)-benzenesulfonamide

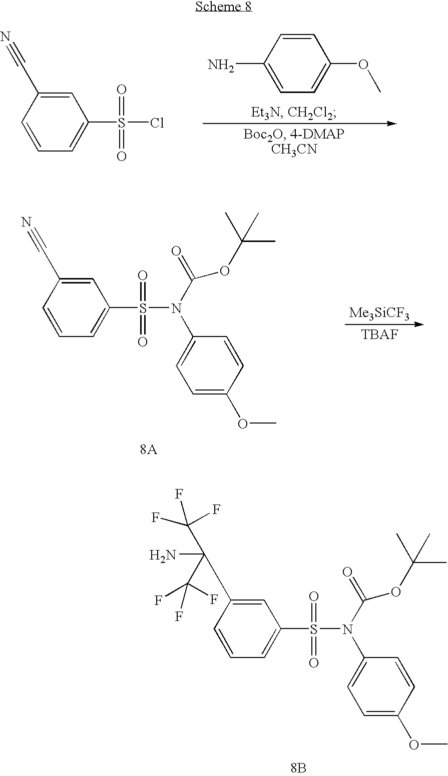

### Step A. 3-Cyano-N-(tert-butoxycarbonyl)-N-(4-methoxy-phenyl)-benzenesulfonamide

The title compound was prepared according to a procedure similar to that described in Example 5, Step A. 3-Cyano-benzenesulfonyl chloride (250 mg, 1.24 mmol) was converted to the desired product (408.9 mg, 85%) as a white solid.

¹H NMR (400 MHz, CDCl₃): δ 1.53 (s, 9H), 3.85 (s, 3H), 6.94 (d, J=8.84 Hz, 2H), 7.12 (d, J=8.84 Hz, 2H), 7.71 (dd, J=8.34, 8.34 Hz, 1H), 7.93 (d, J=8.34 Hz, 1H), 8.22 (d, J=8.34 Hz, 1H), 8.29 (s, 1H).

Step B. The title compound of Example 8 was prepared according to a procedure similar to that described in Example 1, Step B. 3-Cyano-N-(tert-butoxycarbonyl)-N-(4-methoxy-phenyl)-benzenesulfonamide 8A (225 mg, 0.58 mmol) was converted to the desired product (120.0 mg, 39%) as a colorless oil.

¹H NMR (400 MHz, CDCl₃): δ 1.32 (s, 9H), 2.23 (s, 2H), 3.84 (s, 3H), 6.93 (d, J=8.84 Hz, 2H), 7.12 (d, J=8.84 Hz, 2H), 7.66 (dd, J=8.08, 8.08 Hz, 1H), 8.07 (d, J=8.08 Hz, 1H), 8.13 (d, J=8.08 Hz, 1H), 8.41 (s, 1H).

### Example 9

### [4-(1-Amino-2,2,2-trifluoro-1-trifluoromethyl-ethyl)-phenyl]-piperidin-1-yl-methanone

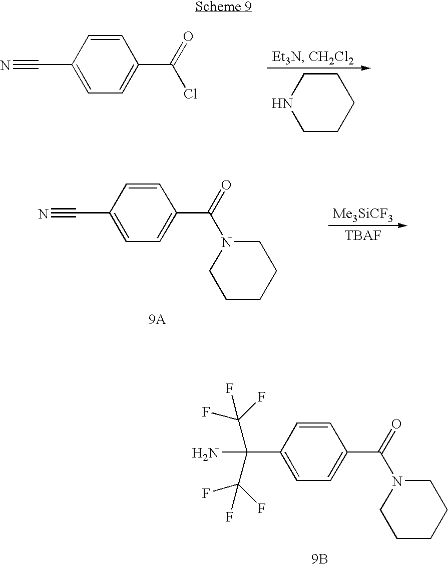

### Step A. 4-(Piperidine-1-carbonyl)-benzonitrile

The title compound was prepared according to a procedure similar to that described in Example 1, Step A. 4-Cyano-benzoyl chloride (100 mg, 0.60 mmol) was converted to the desired product (110 mg, 85%) as a mixture of two isomers in a 1:1 ratio. White solid.

¹H NMR (400 MHz, CDCl₃): δ 1.49-1.56 (m, 2H), 1.66-1.75 (m, 4H), 3.24-3.34 (m, 2H), 3.67-3.76 (m, 2H), 7.49 (d, J=8.59 Hz, 2H), 7.71 (d, J=8.59 Hz, 2H).

Step B. The title compound of Example 9 was prepared according to a procedure similar to that described in Example 1, Step B. 4-(Piperidine-1-carbonyl)-benzonitrile 9A (110 mg, 0.51 mmol) was converted to the desired product (79.0 mg, 44%) as a mixture of two isomers in a 1:1 ratio. White solid.

¹H NMR (400 MHz, CDCl₃): δ 1.49-1.57 (m, 2H), 1.62-1.74 (m, 4H), 2.20 (s, 2H), 3.26-3.40 (m, 2H), 3.68-3.77 (m, 2H), 7.47 (d, J=8.84 Hz, 2H), 7.81 (d, J=8.84 Hz, 2H).

### Example 10

### 4-(1-Amino-2,2,2-trifluoro-1-trifluoromethyl-ethyl)-N-(tert-butoxycarbonyl)-N-(4-methoxy-phenyl)-benzenesulfonamide

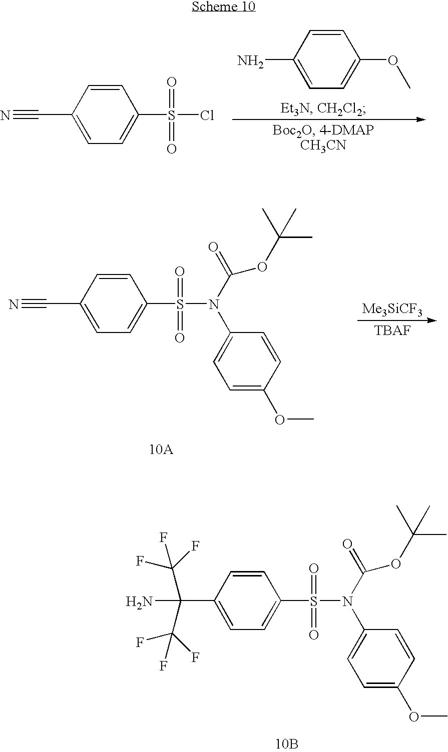

### Step A. 4-Cyano-N-(tert-butoxycarbonyl)-N-(4-methoxy-phenyl)-benzenesulfonamide

The title compound was prepared according to a procedure similar to that described in Example 5, Step A. 4-Cyano-benzenesulfonyl chloride (125 mg, 0.622 mmol) was converted to the desired product (212.4 mg, 88%) as a white solid.

¹H NMR (400 MHz, CDCl₃): δ 1.34 (s, 9H), 3.85 (s, 3H), 6.94 (d, J=8.84 Hz, 2H), 7.12 (d, J=8.84 Hz, 2H), 7.85 (d, J=8.84 Hz, 2H), 8.11 (d, J=8.84 Hz, 2H).

Step B. The title compound of Example 10 was prepared according to a procedure similar to that described in Example 1, Step B. 4-Cyano-N-(tert-butoxycarbonyl)-N-(4-methoxy-phenyl)-benzenesulfonamide 10A (363 mg, 0.935 mmol) was converted to the desired product (250.0 mg, 51%) as a white solid.

¹H NMR (400 MHz, CDCl₃): δ 1.32 (s, 9H), 2.23 (s, 2H), 3.84 (s, 3H), 6.95 (d, J=8.84 Hz, 2H), 7.18 (d, J=9.09 Hz, 2H), 7.99 (d, J=8.84 Hz, 2H), 8.07 (d, J=9.09 Hz, 2H).

### Example 11

### 3-(1-Amino-2,2,2-trifluoro-1-trifluoromethyl-ethyl)-N,N-diethyl-benzamide

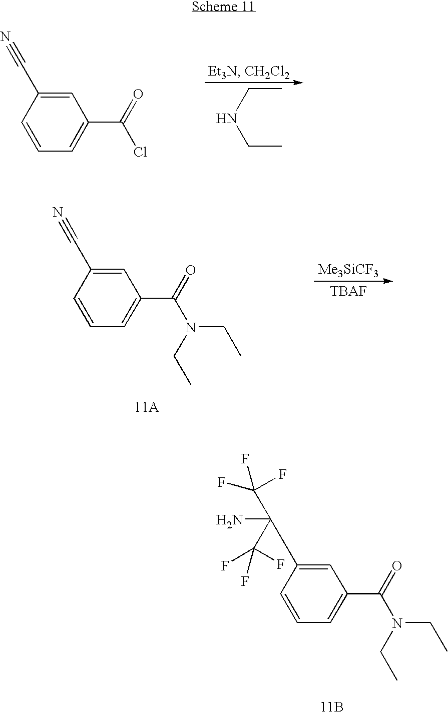

### Step A. 3-Cyano-N,N-diethyl-benzamide

The title compound was prepared according to a procedure similar to that described in Example 1A. 3-Cyano-benzoyl chloride (190 mg, 1.15 mmol) was converted to the desired product (230 mg, 99%) as a mixture of two isomers in a 1:1 ratio. White solid.

¹H NMR (400 MHz, CDCl₃): δ 1.10-1.19 (m, 3H), 1.22-1.31 (m, 3H), 3.20-3.28 (m, 2H), 3.53-3.60 (m, 2H), 7.53 (dd, J=7.83, 7.83 Hz, 1H), 7.62 (d, J=7.83 Hz, 1H), 7.67 (s, 1H), 7.70 (d, J=7.83 Hz, 1H).

Step B. The title compound of Example 11 was prepared according to a procedure similar to that described in Example 1, Step B. 3-Cyano-N,N-diethyl-benzamide 11A (230 mg, 1.13 mmol) was converted to the desired product (231.0 mg, 60%) as a mixture of two isomers in a 1:1 ratio. White solid.

¹H NMR (400 MHz, CDCl₃): δ 1.09-1.14 (m, 3H), 1.23-1.28 (m, 3H), 2.22 (s, 2H), 3.16-3.24 (m, 2H), 3.52-3.59 (m, 2H), 7.47-7.50 (m, 2H), 7.79-7.82 (m, 2H).

### Example 12

### 4-(1-Amino-2,2,2-trifluoro-1-trifluoromethyl-ethyl)-N,N-diethyl-benzamide

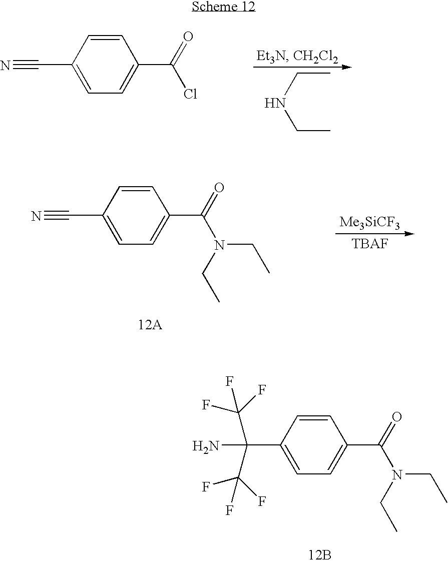

### Step A. 4-Cyano-N,N-diethyl-benzamide

The title compound was prepared according to a procedure similar to that described in Example 1, Step A. 4-Cyano-benzoyl chloride (200 mg, 1.21 mmol) was converted to the desired product (220 mg, 90%) as a mixture of two isomers in a 1:1 ratio. White solid.

¹H NMR (400 MHz, CDCl₃): δ 1.13 (t, J=6.32 Hz, 3H), 1.26 (t, J=6.32 Hz, 3H), 3.18-3.25 (m, 2H), 3.52-3.60 (m, 2H), 7.48 (d, J=8.59 Hz, 2H), 7.71 (d, J=8.59 Hz, 2H).

Step B. The title compound of Example 12 was prepared according to a procedure similar to that described in Example 1, Step B. 4-Cyano-N,N-diethyl-benzamide 12A (220 mg, 1.09 mmol) was converted to the desired product (141.0 mg, 38%) as a mixture of two isomers in a 1:1 ratio. White solid.

¹H NMR (400 MHz, CDCl₃): δ 1.13 (t, J=6.50 Hz, 3H), 1.26 (t, J=6.50 Hz, 3H), 2.20 (s, 2H), 3.25 (q, J=6.50 Hz, 2H), 3.56 (q, J=6.50 Hz, 2H), 7.45 (d, J=8.34 Hz, 2H), 7.81 (d, J=8.34 Hz, 2H).

### Example 13

### 4-(1-Amino-2,2,2-trifluoro-1-trifluoromethyl-ethyl)-N,N-diethyl-benzenesulfonamide

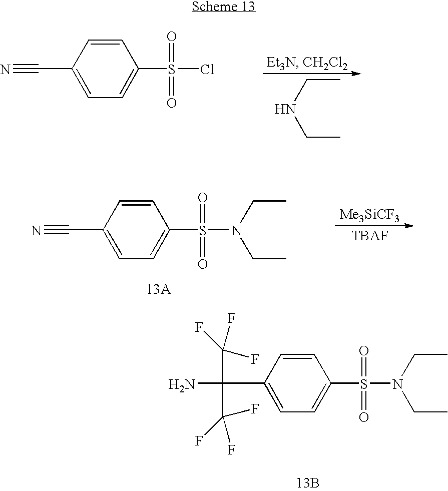

### Step A. 4-Cyano-N,N-diethyl-benzenesulfonamide

The title compound was prepared according to a procedure similar to that described in Example 1, Step A. 4-Cyano-benzenesulfonyl chloride (240 mg, 1.19 mmol) was converted to the desired product (281 mg, 99%) as a white solid.

¹H NMR (400 MHz, CDCl₃): δ 1.15 (t, J=7.07 Hz, 6H), 3.28 (q, J=7.07 Hz, 4H), 7.80 (d, J=8.84 Hz, 2H), 7.93 (d, J=8.84 Hz, 2H).

Step B. The title compound of Example 13 was prepared according to a procedure similar to that described in Example 1, Step B. 4-Cyano-N,N-diethyl-benzenesulfonamide 13A (300 mg, 1.26 mmol) was converted to the desired product (123.0 mg, 26%) as a colorless oil. Note that the final product was isolated via HPLC under neutral conditions.

¹H NMR (400 MHz, CDCl₃): δ 1.14 (t, J=7.07 Hz, 6H), 2.27 (s, 2H), 3.27 (q, J=7.07 Hz, 4H), 7.87 (d, J=8.84 Hz, 2H), 7.94 (d, J=8.84 Hz, 2H).

### Example 14

### 2-(3-{[(2R)-4-{6-[1-amino-2,2,2-trifluoro-1-(trifluoromethyl)ethyl]pyridin-3-yl}-2-methylpiperazin-1-yl]sulfonyl}phenyl)-1,1,1-trifluoropropan-2-ol

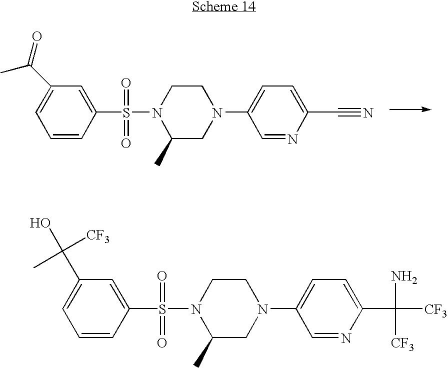

Step 1A: A mixture of (R)-2-methyl-piperazine (1.0 g, 9.98 mmol), 5-bromo 2-cyanopyridine (1.66 g, 9.08 mmol), tris(dibenzylidineacetone)dipalldium (0) (83.15 mg, 0.0908 mmol), rac-2,2′-bis(diphenylphosphino)-1,1′-binaphtyl (169.37 mg, 0.272 mmol) and sodium tert-butoxide (1.09 g, 11.35 mmol) were charged to a microwave vial. Toluene (10.0 mL) was introduced under nitrogen atmosphere and the reaction mixture was irradiated at 110° C. for 35 minutes. Reaction was complete as determined by TLC. Reaction mixtures was diluted with dichloromethane, washed with water, saturated brine then dried over Na₂SO₄ and concentrated. The crude product was purified via flash column chromatography to yield 5-[(3R)-3-methylpiperazin-1-yl]pyridine-2-carbonitrile as brown color oil (1.15 g, 39.1% yield).

Step 1B: To a stirred solution of 5-[(3R)-3-methylpiperazin-1-yl]pyridine-2-carbonitrile (250 mg, 1.24 mmol) and 3-acetylbenzenesulfonyl chloride (270.3 mg, 1.24 mmol) in anhydrous dichloromethane (4 mL) was added diisopropylethylamine (0.43 mL, 2.48 mmol). The mixture was stirred at room temperature for over night. Reaction was complete as determined by TLC. The reaction mixture was purified via flash column chromatography to yield 5-{(3R)-4-[(3-acetylphenyl)sulfonyl]-3-methylpiperazin-1-yl}pyridine-2-carbonitrile in 80.3% yield (383 mg) as a light yellow solid.

Step 1C: To a 50 mL flask containing 5-{(3R)-4-[(3-acetylphenyl)sulfonyl]-3-methylpiperazin-1-yl}pyridine-2-carbonitrile (383 mg, 0.996 mmol) and 6.0 mL of 0.5 M TMS—CF₃, was added 0.996 mL of 1.0 M tetrabutylammonium fluoride in THF at 0° C. After stirring for 2 h, the solution was diluted with saturated NaHCO₃, extracted (2×CH₂Cl₂), washed with brine and dried over Na₂SO₄, and concentrated under reduced pressure. Purification by flash column chromatography to yield 2-(3-{[(2R)-4-{6-[1-amino-2,2,2-trifluoro-1-(trifluoromethyl)ethyl]pyridin-3-yl}-2-methylpiperazin-1-yl]sulfonyl}phenyl)-1,1,1-trifluoropropan-2-ol as a light yellow solid.

HRMS: calcd for C₂₂H₂₃F₉N₄O₃S+H+, 595.14199; found (ESI-FTMS, [M+H]¹⁺), 595.14231.

### Example 15

### 2-(3-{[(2R)-4-{4-[1-amino-2,2,2-trifluoro-1-(trifluoromethyl)ethyl]-2-(trifluoromethyl)phenyl}-2-methylpiperazin-1-yl]sulfonyl}phenyl)-1,1,1-trifluoropropan-2-ol

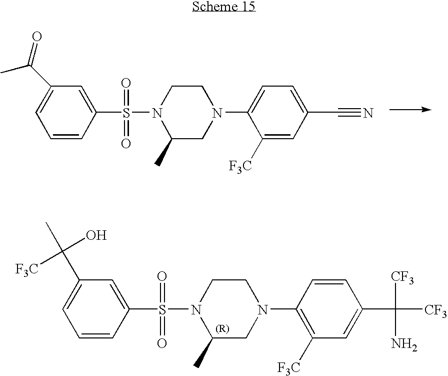

The title compound of Example 15 was prepared according to a procedure similar to that described in Example 14. HRMS: calcd for C₂₄H₂₃F₁₂N₃O₃S+H+, 662.13412; found (ESI-FTMS, [M+H]¹⁺), 662.13513.

### Example 16

### 2-{4-([(2R)-4-[4-[1-amino-2,2,2-trifluoro-1-(trifluoromethyl)ethyl]-2-(trifluoromethyl)phenyl]-2-methylpiperazin-1-yl}sulfonyl)phenyl]-1,1,1-trifluoropropan-2-ol

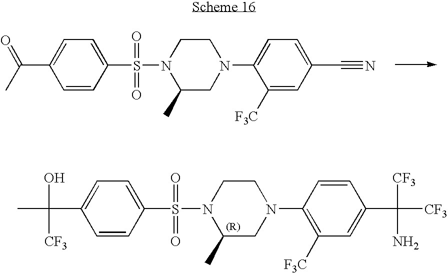

The title compound was prepared according to a procedure similar to that described in Example 14. HRMS: calcd for C₂₄H₂₃F₁₂N₃O₃S+H+, 662.13412; found (ESI-FTMS, [M+H]¹⁺), 662.13495.

### Example 17

### 1,1,1,3,3,3-hexafluoro-2-[4-({(2R)-4-[4-fluoro-2-(trifluoromethyl)-phenyl]-2-methylpiperazin-1-yl}sulfonyl)phenyl]propan-2-amine

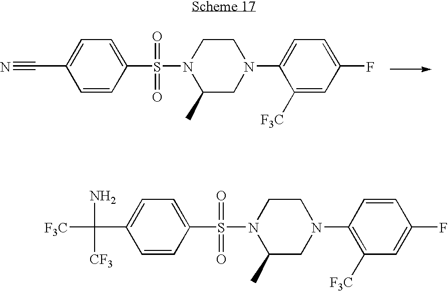

The title compound was prepared according to a similar procedure for Example 14. In step 1B, 4-cyanobenzenesulfonyl chloride was used as starting material to make intermediate. HRMS: calcd for C₂₁H₁₉F₁₀N₃O₂S+H+, 568.11110; found (ESI-FTMS, [M+H]¹⁺), 568.11129.

### Example 18

### 1,1,1,3,3,3-hexafluoro-2-[3-({(2R)-4-[4-fluoro-2-(trifluoromethyl)-phenyl]-2-methylpiperazin-1-yl}sulfonyl)phenyl]propan-2-amine

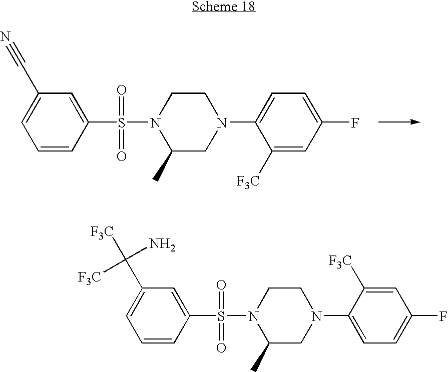

The title compound was prepared according to a similar procedure for Example 14. In step 1B, 3-cyanobenzenesulfonyl chloride was used as starting material to make intermediate. for C₂₁H₁₉F₁₀N₃O₂S+H+, 568.11110; found (ESI-FTMS, [M+H]¹⁺), 568.11142.

### Example 19

### Biological Testing

Compounds described herein can be tested in a cell-based assay using a stable CHO cell line expressing human 11b-HSD1. Cells are plated at 20,000 cells/well in 96 well plates and incubated overnight (12-16 hrs) at 37° C./5% CO₂. Cells are treated with different concentration of compound in 90 microliter serum-free media and incubated for 30 minutes at 37° C./5% CO₂. 10 ul of 5 micromolar cortisone (final concentration 500 nM) is then added to the cells and the plate is incubated at 37° C./5% CO₂ for 120 minutes. 15 microliter of media is withdrawn and amount of cortisol in the media is measured using the DiscoverX HitHunter Cortisol Assay (DiscoverX corp, CA).

To determine the potency of compounds against mouse 11b-HSD1, a stable CHO cell line expressing mouse 11b-HSD1 is used. Cells are plated at 20,000 cells/well in 96 well plates and incubated overnight (12-16 hrs) at 37° C./5% CO₂. Cells are treated with different concentration of compound in 90 microliter serum-free media and incubated for 30 minutes at 37° C./5% CO₂. To determine the potency of the compound against mouse 11-bHSD 1 in the presence of serum, 90 microliter media containing 10% delipidized human serum is used instead of serum free media. 10 ul of 5 micromolar cortisone (final concentration 500 nM) is then added to the cells and the plate is incubated at 37° C./5% CO₂ for 120 minutes. 15 microliter of media is withdrawn and amount of cortisol in the media is measured using the DiscoverX HitHunter Cortisol Assay (DiscoverX corp, CA).

Results:

| | Human | Mouse | Mouse + Serum |
|---|---|---|---|
| Example | 1C50 (μM) | 1C50 (μM) | 1C50 (μM) |
| 1 | >10 | >10 | 3.321 |
| 2 | 0.428 | 0.613 | 0.731 |
| 3 | >10 | >10 | >10 |
| 4 | >10 | >10 | >10 |
| 5 | >10 | >10 | >10 |
| 6 | >10 | >10 | >10 |
| 7 | 2.377 | >10 | 2.692 |
| 8 | >10 | >10 | >10 |
| 9 | >10 | >10 | 8.382 |
| 10 | >10 | >10 | >10 |
| 11 | — | — | 0.291 |
| 12 | — | — | 0.165 |
| 13 | 0.565 | 0.225 | 0.645 |
| 14 | <0.01 | 0.160 | — |
| 15 | 0.080 | 0.200 | — |
| 16 | 0.090 | <0.1 | — |
| 17 | 0.170 | 0.013 | — |

A number of embodiments of the invention have been described. Nevertheless, it will be understood that various modifications may be made without departing from the spirit and scope of the invention. Accordingly, other embodiments are within claims.

What is claimed is:

1. A method for preparing an organic compound or a salt thereof having one or more substituents of formula (A): 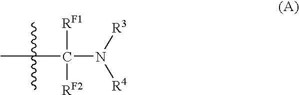
wherein:
(i) each of R^{F1} and R^{F2} is, independently, optionally substituted C₁-C₆ fluoroalkyl;
(ii) each of R³ and R⁴ is, independently, hydrogen, R^{a}, —C(O)H, —C(O)R^{a}, —C(O)OR^{a}, or —SO₂R^{a}, wherein R^{a} at each occurrence is, independently, any organic group, selected from alkyl, cycloalkyl, aralkyl, heterocyclyl, aryl, or heteroaryl, each of which is optionally substituted; and
(iii) the organic compound comprises as part of its structure any one or more of the following substructures:
(i) C₆-C₁₈ aryl or heteroaryl including 5-16 atoms, each of which is optionally substituted; or
(ii) C₇-C₂₀ aralkyl or heteroaralkyl including 6-20 atoms, each of which is optionally substituted; or
(iii) C₃-C₁₀ cycloalkyl, C₃-C₁₀ cycloalkenyl, heterocyclyl including 3-10 atoms, or heterocycloalkenyl including 3-10 atoms, each of which is optionally substituted; or
(iv) C₁-C₁₂ alkyl, C₂-C₁₂ alkenyl or C₂-C₁₂ alkynyl, each of which is optionally substituted;
each of which, when two or more substructures are present, is connected to one another by a direct bond or a heteroatom-containing linker;
the method comprising reacting a nitrile-containing organic compound with a fluoroalkylating agent.

2. The method of claim 1, wherein R^{F1} and R^{F2} are the same.

3. The method of claim 1, wherein each of R^{F1} and R^{F2} is, independently, optionally substituted C₁-C₄ perfluoroalkyl.

4. The method of claim 1, wherein each of R^{F1} and R^{F2} is CF₃.

5. The method of claim 1, wherein each of R³ and R⁴ is hydrogen.

6. The method of claim 1, wherein the organic compound having one or more substituents of formula (A) is a compound of formula (I) or a salt thereof: 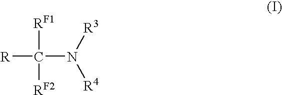
wherein:
R is:
(i) C₆-C₁₈ aryl or heteroaryl including 5-16 atoms, each of which is optionally substituted;
(ii) C₇-C₂₀ aralkyl or heteroaralkyl including 6-20 atoms, each of which is optionally substituted; or
(iii) C₃-C₁₀ cycloalkyl, C₃-C₁₀ cycloalkenyl, heterocyclyl including 3-10 atoms, or heterocycloalkenyl including 3-10 atoms, each of which is optionally substituted; or
(iv) C₁-C₁₂ alkyl, C₂-C₁₂ alkenyl or C₂-C₁₂ alkynyl, each of which is optionally substituted;
each of R^{F1} and R^{F2} is, independently, optionally substituted C₁-C₆ fluoroalkyl; and
each of R³ and R⁴ is, independently, hydrogen, R^{a}, —C(O)H, —C(O)R^{a}, —C(O)OR^{a}, or —SO₂R^{a}, wherein R^{a} at each occurrence is, independently, as defined above for R;
and the nitrile-containing organic compound is a compound of formula (II): 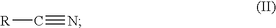
wherein R is as defined above.

7. The method of claim 6, wherein R is optionally substituted C₆-C₁₀ aryl.

8. The method of claim 6, wherein R is optionally substituted phenyl.

9. The method of claim 6, wherein R is optionally substituted C₇-C₁₂ aralkyl.

10. The method of claim 6, wherein R is optionally substituted benzyl.

11. The method of claim 1, wherein the fluoroalkylating agent is a perfluoroalkylating agent.

12. The method of claim 1, wherein the fluoroalkylating agent is a trifluoromethylating agent.

13. The method of claim 1, wherein the fluoroalkylating agent is a compound having formula (III): 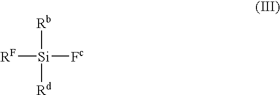
wherein:
R^{F} is C₁-C₆ fluoroalkyl; and
each of R^{b}, R^{c}, and R^{d} is, independently, C₁-C₁₂ alkyl or C₂-C₁₂ alkenyl, each of which is optionally substituted.

14. The method of claim 13, wherein a fluoride ion is present during the reacting of the compound of formula (II) and the compound of formula (III).

15. The method of claim 13, wherein each of R^{b}, R^{c}, and R^{d} is, independently, C₁-C₄ alkyl.

16. The method of claim 13, wherein each of R^{b}, R^{c}, and R^{d} is —CH₃.

17. The method of claim 13, wherein each of R^{b}, R^{c}, and R^{d} is —CH₂CH₃.

18. The method of claim 13, wherein one of R^{b}, R^{c}, and R^{d} is C₂-C₄ alkenyl, and the other two are each, independently, C₁-C₄ alkyl.

19. The method of claim 13, wherein one of R^{b}, R^{c}, and R^{d} is —CH═CH₂, and the other two are each —CH₃.

20. The method of claim 1, wherein the fluoroalkylating agent is a fluoroalkyl-containing salt or ionic complex.

21. The method of claim 20, wherein the method further comprises reacting a compound having formula (IV):
R^{F}—X
wherein R^{F} is C₁-C₆ fluoroalkyl; and X is halo;
with a reducing agent.

22. The method of claim 21, wherein the reducing agent is tetrakis(dimethylamino)ethylene (TDAE).

23. The method of claim 21, wherein X is iodo.

24. The method of claim 21, wherein R^{F} is CF₃, CF₂CF₃, or (CF₂)₃CF₃.

25. The method of claim 1, wherein the fluoroalkylating agent is a compound having formula (V): 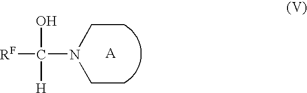
wherein:
R_{F} is C₁-C₆ fluoroalkyl; and
ring A is optionally substituted morpholinyl or piperazinyl.

26. The method of claim 25, wherein a base is present during the reacting of the compound of formula (II) and the compound of formula (V).

27. The method of claim 26, wherein the base is a metal salt of a C₁-C₆ alkoxide.

28. The method of claim 25, wherein R^{F} is CF₃.

29. The method of claim 1, wherein the fluoroalkylating agent is a compound having formula (VI): Ar—S(O)ₓ—R^{F};
wherein:
Ar is optionally substituted phenyl;
x is 1 or 2; and
R^{F} is C₁-C₆ fluoroalkyl.

30. The method of claim 29, wherein a base is present during the reacting of the compound of formula (II) and the compound of formula (VI).

31. The method of claim 30, wherein the base is a metal salt of a C₁-C₆ alkoxide.

32. The method of claim 29, wherein R_{F} is CF₃.

33. The method of claim 29, wherein x is 2.

34. The method of claim 1, wherein the fluoroalkylating agent is CF₃H.

35. The method of claim 1, wherein the compound of formula (II) further comprises a substituent having a formula —C(O)R^{e}, wherein R^{e} is C₁-C₆ alkyl.

36. The method of claim 35, wherein R^{e} is CH₃.

37. A method for preparing a compound of formula (VII) or a salt thereof: 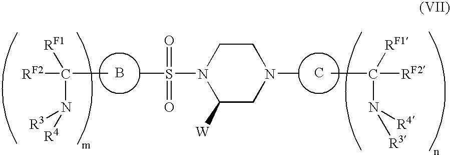
wherein:
each of m and n is, independently, 0 or 1, provided that one of m and n is 1;
each of R^{F1}, R^{F2}, R^{F1′}, and R^{F2′} is, independently, optionally substituted C₁-C₆ fluoroalkyl;
each of R³, R⁴, R^{3′}, and R^{4′} is, independently, hydrogen, C₁-C₆ alkyl, —C(O)H, or —C(O)OR^{a}, wherein R^{a} is C₇-C₂₀ aralkyl or C₁-C₆ alkyl, each of which is optionally substituted;
ring B is C₆-C₁₀ aryl or heteroaryl including 5-10 atoms, each of which is optionally further substituted with from 1-5 substituents independently selected from halo; NR^{f}R^{g}; hydroxyl; C₁-C₁₂ alkyl or C₁-C₁₂ haloalkyl, each of which is optionally substituted; optionally substituted C₁-C₁₂ alkoxy; C₁-C₁₂ haloalkoxy; nitro; C₆-C₁₀ aryl or heteroaryl including 5-12 atoms, each of which is optionally substituted; C₆-C₁₀ aryloxy or heteroaryloxy including 5-12 atoms, each of which is optionally substituted; heterocyclyl including 3-10 atoms, C₃-C₁₀ cycloalkyl, C₇-C₁₂ aralkoxy or heteroaralkoxy including 6-12 atoms, each of which is optionally substituted; —C(O)OR^{h}; —C(O)NR^{f}R^{g}; or —NRⁱC(O)R^{j};
each of R^{f}, R^{g}, and R^{h}, at each occurrence is, independently:
(i) hydrogen; or
(ii) C₁-C₁₂ alkyl or C₁-C₁₂ haloalkyl; each of which is optionally substituted; or
(iii) C₇-C₂₀ aralkyl; C₃-C₁₆ cycloalkyl; heteroaralkyl including 6-20 atoms; C₃-C₁₆ cycloalkenyl; heterocyclyl including 3-16 atoms; or heterocycloalkenyl including 3-16 atoms; each of which is optionally substituted; or
(iv) C₂-C₂₀ alkenyl or C₂-C₂₀ alkynyl; or
(v) C₆-C₁₆ aryl or heteroaryl including 5-16 atoms, each of which is optionally substituted;
R^{j} is R^{h}; OR^{h}; or NR^{f}R^{g};
W is C₁-C₄ alkyl; and
ring C is C₆-C₁₀ aryl or heteroaryl including 5-10 atoms, each of which is optionally further substituted with from 1-5 substituents independently selected from halo; C₁-C₁₂ alkyl or C₁-C₁₂ haloalkyl, each of which is optionally substituted; C₁-C₁₂ alkoxy; C₁-C₁₂ haloalkoxy; nitro; or C₆-C₁₀ aryl or heteroaryl including 5-12 atoms, each of which is optionally substituted;
from a compound of formula (VIII): 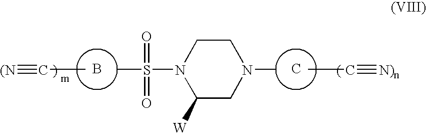
wherein:
ring B is C₆-C₁₀ aryl or heteroaryl including 5-10 atoms, each of which is optionally further substituted with from 1-5 substituents independently selected from halo; NR^{f}R^{g}; hydroxyl; C₁-C₁₂ alkyl or C₁-C₁₂ haloalkyl, each of which is optionally substituted; optionally substituted C₁-C₁₂ alkoxy; C₁-C₁₂ haloalkoxy; nitro; C₆-C₁₀ aryl or heteroaryl including 5-12 atoms, each of which is optionally substituted; C₆-C₁₀ aryloxy or heteroaryloxy including 5-12 atoms, each of which is optionally substituted; heterocyclyl including 3-10 atoms, C₃-C₁₀ cycloalkyl, C₇-C₁₂ aralkoxy or heteroaralkoxy including 6-12 atoms, each of which is optionally substituted; —C(O)R^{e}, wherein R^{e} is C₁-C₆ alkyl; —C(O)NR^{f}R^{g}; or —NRⁱC(O)R^{j}; and
m, n, ring C, and W are as defined above in conjunction with formula (VII);
the method comprising reacting the compound of formula (VIII) with a fluoroalkylating agent.

38. The method of claim 37, wherein m in formulas (VII) and (VIII) is 1, and n in formulas (VII) and (VIII) is 0.

39. The method of claim 38, wherein each of R^{F1} and R^{F2} in formula (VII) is CF₃.

40. The method of claim 38, wherein each of R³ and R⁴ in formula (VII) is hydrogen.

41. The method of claim 38, wherein ring C in formula (VII) has formula (IX): 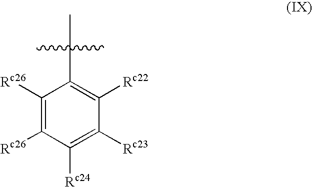
wherein two of R^{c22}, R^{c23}, R^{c24}, R^{c25}, and R^{c26} are each, independently, halo; C₁-C₁₂ alkyl or C₁-C₁₂ haloalkyl, each of which is optionally substituted; C₁-C₁₂ alkoxy; C₁-C₁₂ haloalkoxy; nitro; or C₆-C₁₀ aryl or heteroaryl including 5-12 atoms, each of which is optionally substituted; and the others are hydrogen.

42. The method of claim 41, wherein R^{c22} is CF₃ or fluoro; and R^{c24} is fluoro, chloro, CF₃, or optionally substituted heteroaryl.

43. The method of claim 37, wherein m in formulas (VII) and (VIII) is 0, and n in formulas (VII) and (VIII) is 1.

44. The method of claim 43, wherein each of R^{F1′} and R^{F2′} in formula (VII) is CF₃.

45. The method of claim 43, wherein each of R^{3′} and R^{4′} in formula (VII) is hydrogen.

46. The method of claim 43, wherein ring B in formula (VII) has formula (X): 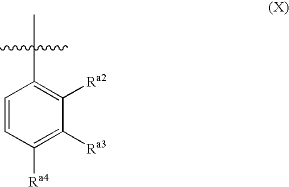
wherein one of R^{a2}, R^{a3}, and R^{a4} is halo; NR^{f}R^{g}; hydroxyl; C₁-C₁₂ alkyl or C₁-C₁₂ haloalkyl, each of which is optionally substituted; optionally substituted C₁-C₁₂ alkoxy; C₁-C₁₂ haloalkoxy; nitro; C₆-C₁₀ aryl or heteroaryl including 5-12 atoms, each of which is optionally substituted; C₆-C₁₀ aryloxy or heteroaryloxy including 5-12 atoms, each of which is optionally substituted; heterocyclyl including 3-10 atoms, C₃-C₁₀ cycloalkyl, C₇-C₁₂ aralkoxy or heteroaralkoxy including 6-12 atoms, each of which is optionally substituted; —C(O)OR^{h}; —C(O)NR^{f}R^{g}; or —NRⁱC(O)R^{j}; and the others are hydrogen.

47. The method of claim 46, wherein R^{a3} or R^{a4} is 1,1,1-trifluoro-2-hydroxy-2-propyl.

48. The method of claim 43, wherein ring B in formula (VIII) is substituted with —C(O)R^{e}, wherein R^{e} is C₁-C₄ alkyl.

49. The method of claim 37, wherein the fluoroalkylating agent is a compound having formula (III): 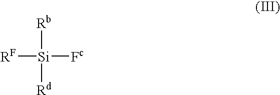
wherein:
R^{F} is C₁-C₆ fluoroalkyl; and
each of R^{b}, R^{c}, and R^{d} is, independently, C₁-C₁₂ alkyl or C₂-C₁₂ alkenyl, each of which is optionally substituted.

50. The method of claim 49, wherein a fluoride ion is present during the reacting of the formula (VII) and the compound of formula (III).

51. The method of claim 49, wherein each of R^{b}, R^{c}, and R^{d} is, independently, C₁-C₄ alkyl.

52. The method of claim 49, wherein each of R^{b}, R^{c}, and R^{d} is —CH₃.

53. The method of claim 49, wherein each of R^{b}, R^{c}, and R^{d} is —CH₂CH₃.

54. The method of claim 49, wherein one of R^{b}, R^{c}, and R^{d} is C₂-C₄ alkenyl, and the other two are each, independently, C₁-C₄ alkyl.

55. The method of claim 49, wherein one of R^{b}, R^{c}, and R^{d} is —CH═CH₂, and the other two are each —CH₃.

56. A compound selected from the group consisting of:
2-(3-{[(2R)-4-{6-[1-amino-2,2,2-trifluoro-1-(trifluoromethyl)ethyl]pyridin-3-yl}-2-methylpiperazin-1-yl]sulfonyl}phenyl)-1,1,1-trifluoropropan-2-ol;
2-(3-{[(2R)-4-{4-[1-amino-2,2,2-trifluoro-1-(trifluoromethyl)ethyl]-2-(trifluoromethyl)phenyl}-2-methylpiperazin-1-yl]sulfonyl}phenyl)-1,1,1-trifluoropropan-2-ol;
2-[4-({(2R)-4-[4-[1-amino-2,2,2-trifluoro-1-(trifluoromethyl)ethyl]-2-(trifluoromethyl)phenyl]-2-methylpiperazin-1-yl}sulfonyl)phenyl]-1,1,1-trifluoropropan-2-ol;
1,1,1,3,3,3-hexafluoro-2-[4-({(2R)-4-[4-fluoro-2-(trifluoromethyl)phenyl]-2-methylpiperazin-1-yl}sulfonyl)phenyl]propan-2-amine; and
1,1,1,3,3,3-hexafluoro-2-[3-({(2R)-4-[4-fluoro-2-(trifluoromethyl)phenyl]-2-methylpiperazin-1-yl}sulfonyl)phenyl]propan-2-amine; or a pharmaceutically acceptable salt thereof.

57. A compound selected from the group consisting of the title compounds of Examples 1-13, or a pharmaceutically acceptable salt thereof.
